(19) United States
(12) Patent Application Publication
(10) Pub. No.: US 2026/0115331 A1
(43) Pub. Date: Apr. 30, 2026

(54) PET IMAGING OF BACTERIAL INFECTION WITH A PET PROBE

(71) Applicant: Mayo Foundation for Medical Education and Research, Rochester, MN (US)

(72) Inventors: Mukesh K. Pandey, Rochester, MN (US); Aditya Bansal, Rochester, MN (US); Val J. Lowe, Rochester, MN (US); Robin Patel, Rochester, MN (US)

(21) Appl. No.: 19/116,756

(22) PCT Filed: Sep. 28, 2023
(86) PCT No.: PCT/US2023/075339

§ 371 Date: Mar. 28, 2025

Related U.S. Application Data
(60) Provisional application No. 63/411,705, filed on Sep. 30, 2022.

(51) Int. Cl. A61K 51/02

(52) U.S. Cl. CPC A61K 51/02; A61K 2123/00

(57) ABSTRACT

Disclosed are compositions and methods for the imaging of bacterial infection and differentiation of bacterial infection with inflammation and presence of bacterial in the host body. One embodiment of the composition comprises a radiopharmaceutical including: (i) cations comprising a positron emitter, and (ii) anions selected from the group consisting of [PO₄]³⁻, [HPO₄]²⁻, [H₂PO₄]⁻, and mixtures thereof, wherein the cations are ionically bonded to the anions. One embodiment of the method for in vivo imaging of a subject, comprises: (a) administering to the subject the composition; (b) waiting a time sufficient to allow the radiopharmaceutical to accumulate at a tissue or cell site or an organ of interest to be imaged; (c) imaging the cells or tissues or the organ of interest with a medical imaging technique; and (d) imaging of infected bone or any foreign body object (devices and implants) with bacteria.

### CROSS-REFERENCES TO RELATED APPLICATIONS

The present application is based on, claims priority to, and incorporates herein by reference in its entirety for all purposes, U.S. Patent Application No. 63/411,705, filed Sep. 30, 2022.

### STATEMENT REGARDING FEDERALLY SPONSORED RESEARCH

Not Applicable.

### BACKGROUND OF THE INVENTION

### 1. Field of the Invention

This invention relates to the use of positron emission tomography (PET) imaging probes and their use in medical imaging of bacterial infection and for differentiation of a bacterial infection from an inflammation.

### 2. Description of the Related Art

Current imaging modalities to detect bacterial infection are only effective once significant morphological change has occurred and the infection is significant.

Therefore, there exists an unmet need to develop an imaging probe for imaging of bacterial infection with greater sensitivity.

### SUMMARY OF THE INVENTION

In one aspect, the present disclosure provides a composition comprising: a radiopharmaceutical including: (i) cations comprising a positron emitter, and (ii) anions selected from the group consisting of [PO₄]³⁻, [HPO₄]²⁻, [H₂PO₄]⁻, and mixtures thereof, wherein the cations are ionically bonded to the anions. The positron emitter can be selected from the group consisting of ¹¹C, ¹³N, ¹⁵O, ¹⁸F, ^{34m}Cl, ³⁸K, ⁴³Sc, ⁴⁴Sc, ⁴⁵Ti, ⁵¹Mn, ⁵²Mn, ^{52m}Mn, ⁵²Fe, ⁵³Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ^{189/189m}Hg, ¹⁹¹Hg, ^{191m}Hg, ¹⁹³Hg, ^{193m}Hg, ¹⁹⁵Hg, ^{195m}Hg ⁷⁵Br, ⁷⁶Br, ⁸²Rb, ⁸⁶Y, ⁸⁹Zr, ⁹⁰Nb, ^{94m}Tc, ^{110m}In, ¹¹⁵Sb, ^{116m2}Sb, ^{118m2}Sb, ¹¹⁸Sb, ¹²⁰Sb, and ¹²⁴I. The positron emitter can be ⁶⁸Ga. The radiopharmaceutical can have the formula [⁶⁸Ga]Ga-H₃P₂O₈ also known as [⁶⁸Ga]Ga-hydrogen bisphosphate or simply [⁶⁸Ga]Ga-bisphosphate. The radiopharmaceutical can have the following structure:

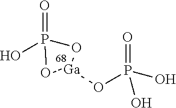

In one embodiment, the composition can further comprise at least one of phosphate buffer, potassium chloride, sodium chloride, and mixtures thereof. In one embodiment, a pH of the composition is in a range of 5 to 7. In one embodiment, the radiopharmaceutical is adapted for targeting a site of bacterial infection. In one embodiment, the radiopharmaceutical is adapted for targeting a site of one of both of gram-negative and gram-positive bacterial infection of any organ or tissue. In one embodiment, the radiopharmaceutical is adapted for targeting a site of one or both of gram-negative and gram-positive bacterial infection of bone, muscle, heart, liver, lung, vascular grafts, interventional grafts, vascular graft infections or foreign body surgical implants.

In one embodiment, a detectable amount of the radiopharmaceutical is present in the composition, and the detectable amount of the radiopharmaceutical is an amount of the radiopharmaceutical that is sufficient to enable detection of accumulation of the radiopharmaceutical in cells or tissue or an organ of interest at a site of bacterial infection by a medical imaging technique. In one embodiment, the medical imaging technique is selected from positron emission tomography imaging, positron emission tomography with computed tomography imaging, positron emission tomography with magnetic resonance imaging, or positron emission tomography with single-photon emission computed tomography. In one embodiment, the medical imaging technique is positron emission tomography imaging.

In one embodiment, the site of bacterial infection is a bone or adjacent to a bone or graft infection. In one embodiment, the site of bacterial infection is a femur or a thigh bone or a bacterial infection related to orthopedic surgical implants. In one embodiment, the site of bacterial infection is a surgical implant selected from spine infusion, a hip replacement, and a knee replacement. In one embodiment, the bacterial infection is a result of a gram-negative bacteria. In one embodiment, the bacterial infection is a result of Escherichia coli. In one embodiment, the bacterial infection is a result of a gram-positive bacteria. In one embodiment, the bacterial infection is a result of Staphylococcus aureus.

In another aspect, the present disclosure provides a method for in vivo imaging of a subject. The method can comprise: (a) administering to the subject the composition of the present disclosure; (b) waiting a time sufficient to allow the radiopharmaceutical of the composition to accumulate at a tissue or cell site or an organ of interest to be imaged; and (c) imaging the cells or tissues or the organ of interest with a medical imaging technique. The medical imaging technique can be selected from positron emission tomography imaging, positron emission tomography with computed tomography imaging, positron emission tomography with magnetic resonance imaging, or positron emission tomography with single-photon emission computed tomography.

In another aspect, the present disclosure provides a method of imaging a subject by positron emission tomography. The method can comprise: (a) administering the composition of the present disclosure to the subject; (b) using a plurality of detectors to detect gamma rays emitted from the subject and to communicate signals corresponding to the detected gamma rays; and (c) reconstructing from the signals a series of medical images of a region of interest of the subject.

In another aspect, the present disclosure provides an imaging method. The method can comprise acquiring an image of a subject to whom a detectable amount of the radiopharmaceutical of the composition of the present disclosure has been administered. In one embodiment, the method comprises acquiring an image of a region of bacterial infection in a bone or adjacent to a bone in a muscle of the subject. The region of bacterial infection can be in or adjacent to a femur, a thigh bone, or a surgical implant of the subject. In one embodiment, the method comprises acquiring an image of a region of a heart or adjacent to a heart of the subject. In one embodiment, the method comprises acquiring an image of a region of bacterial infection related to myocardial infection or a cardiac device. In one embodiment, the method comprises acquiring an image of a region of bacterial infection related to a pacemaker. In one embodiment, the method comprises acquiring an image of a region of a joint or adjacent to a joint of the subject. The joint can be arthritic or include an orthopedic surgical implant. In one embodiment, the method comprises acquiring the image using positron emission tomography imaging, positron emission tomography with computed tomography imaging, positron emission tomography with magnetic resonance imaging, or positron emission tomography with single-photon emission computed tomography. In one embodiment, the detectable amount of the radiopharmaceutical is an amount of the radiopharmaceutical that is sufficient to enable detection of accumulation of the radiopharmaceutical in cells or tissue or an organ of interest by a medical imaging technique.

In another aspect, the present disclosure provides a method for detecting bacterial infection in a subject. The method can comprise: (a) administering to the subject the composition of the present disclosure; (b) waiting a time sufficient to allow the radiopharmaceutical to accumulate at a tissue or cell site or an organ of interest to be imaged; and (c) imaging the cells or tissues or the organ of interest with a medical imaging technique. The method may further comprise (d) comparing a data set from the image of the cells or tissues or the organ of interest to a previously acquired data set from an image of a known infection. In one embodiment, the bacterial infection is a result of a gram-negative bacteria. In one embodiment, the bacterial infection is a result of Escherichia coli. In one embodiment, the bacterial infection is a result of a gram-positive bacteria. In one embodiment, the bacterial infection is a result of Staphylococcus aureus. In one embodiment, the tissue or cell site is a bone or adjacent to a bone or grafts. In one embodiment, the bone is a femur or a thigh of the subject. In one embodiment, the medical imaging technique is selected from positron emission tomography imaging, positron emission tomography with computed tomography imaging, positron emission tomography with magnetic resonance imaging, or positron emission tomography with single-photon emission computed tomography. In one embodiment, step (b) comprises allowing the radiopharmaceutical to accumulate at a site of bacterial infection. In one embodiment, step (b) comprises allowing the radiopharmaceutical to accumulate at a site of bacterial infection including gram-positive bacteria. In one embodiment, step (b) comprises allowing the radiopharmaceutical to accumulate at a site of bacterial infection including gram-negative bacteria.

In another aspect, the present disclosure provides a method for detecting or ruling out a condition involving a bacterial infection in a subject. The method may comprise: (a) administering to a subject the composition of the present disclosure wherein the radiopharmaceutical is targeted to a bacterial infection at a tissue or cell site or an organ of interest in the subject; and (b) acquiring an image of the cells or tissues or the organ of interest to detect the presence or absence of a bacterial infection in the subject. The method may further comprise (c) comparing a data set from the image of the cells or tissues or the organ of interest to a previously acquired data set from an image of a known infection. The method may further comprise determining the SUV from the image of the cells or tissues or the organ of interest and comparing the SUV to a previously acquired SUV from an image of a known infection. In one embodiment, step (b) comprises acquiring the image using a medical imaging technique selected from positron emission tomography imaging, positron emission tomography with computed tomography imaging, positron emission tomography with magnetic resonance imaging, and positron emission tomography with single-photon emission computed tomography. In one embodiment, step (b) comprises acquiring an image of a region of bacterial infection in a bone or adjacent to a bone in a muscle of the subject. In one embodiment, the region of bacterial infection is in or adjacent to a femur, a thigh bone, or a surgical implant of the subject. In one embodiment, step (b) comprises acquiring an image of a region of a heart or adjacent to a heart of the subject. In one embodiment, step (b) comprises acquiring an image of a region of bacterial infection related to myocardial infection or a cardiac device. In one embodiment, step (b) comprises acquiring an image of a region of bacterial infection related to a pacemaker. In one embodiment, step (b) comprises acquiring an image of a region of a joint or adjacent to a joint of the subject. In one embodiment, the joint is arthritic or includes an orthopedic surgical implant. In one embodiment, the bacterial infection is a result of a gram-negative bacteria. In one embodiment, the bacterial infection is a result of Escherichia coli. In one embodiment, the bacterial infection is a result of a gram-positive bacteria. In one embodiment, the bacterial infection is a result of Staphylococcus aureus. In one embodiment, the method can differentiate between inflammation and the bacterial infection. In one embodiment, the cation conjugates with ferritin and/or transferrin and/or lactoferrin at the tissue or cell site.

In another aspect, the present disclosure provides a method for detecting or ruling out a condition involving inflammation in a subject. The method may comprise: (a) administering to a subject the composition of the present disclosure wherein the radiopharmaceutical is targeted to a site of inflammation in a tissue or cell site or an organ of interest in the subject; and (b) acquiring an image of the cells or tissues or the organ of interest to detect the presence or absence of an inflammation. The method may further comprise comparing a data set from the image of the cells or tissues or the organ of interest to a previously acquired data set from an image of a known inflammation. The method may further comprise determining the SUV from the image of the cells or tissues or the organ of interest and comparing the SUV to a previously acquired SUV from an image of a known inflammation. In one embodiment, the inflammation is chronic inflammation, neurological inflammation, or inflammation in extremities of the subject. In one embodiment, step (b) comprises acquiring the image using a medical imaging technique selected from positron emission tomography imaging, positron emission tomography with computed tomography imaging, positron emission tomography with magnetic resonance imaging, and positron emission tomography with single-photon emission computed tomography. In one embodiment, step (b) comprises acquiring an image of a region of inflammation in a bone or adjacent to a bone in a muscle of the subject or in a graft or adjacent to the graft. In one embodiment, the region of inflammation is in or adjacent to a femur, a thigh bone, or a surgical implant of the subject. In one embodiment, step (b) comprises acquiring an image of a region of a heart or adjacent to a heart of the subject. In one embodiment, step (b) comprises acquiring an image of a region of inflammation related to myocardial infection or a cardiac device. In one embodiment, step (b) comprises acquiring an image of a region of inflammation related to a pacemaker. In one embodiment, step (b) comprises acquiring an image of a region of a joint or adjacent to a joint of the subject. In one embodiment, the joint is arthritic or includes an orthopedic surgical implant.

It is one advantage of a composition of the present invention that the invented composition shows about 2-fold higher uptake as gallium citrate in a direct comparison.

It is another advantage of a composition of the present invention that the composition takes less than half the time to produce as gallium citrate (about 5-7 minutes vs. about 15 minutes) and may be less expensive to produce than other radiotracers or PET probes in the field.

It is another advantage of a composition of the present invention that the composition uptake level also enables one to differentiate between inflammation and infection. In fact, the developed PET probe can be envisioned to be useful in imaging of inflammation alone as well.

It is another advantage of a composition of the present invention that the composition enables imaging of live bacteria.

These and other features, aspects, and advantages of the present invention will become better understood upon consideration of the following detailed description, drawings, and appended claims.

### BRIEF DESCRIPTION OF THE DRAWINGS

FIG. 1 is a schematic of a positron emission tomography (PET) system.

FIG. 2 shows a chemical structure of [⁶⁸Ga]Ga-H₃P₂O₈.

FIG. 3 shows a chemical structure of disodium phosphate.

FIG. 4 shows an rTLC analysis of [⁶⁸Ga]GaCl₃ in 0.0025 M EDTA (pH 5.2) solution.

FIG. 5 shows an rTLC analysis of [⁶⁸Ga]Ga-H₃P₂O₈ in 0.0025 M EDTA (pH 5.2) solution.

FIG. 6 is an IR spectrum of phosphate (PBS) in water.

FIG. 7 is an IR spectrum of Ga-H₃P₂O₈ in water.

FIG. 8 is a ³¹P-NMR spectrum of PBS (phosphate buffer)

FIG. 9 is a ³¹P-NMR spectrum of Ga-H₃P₂O₈.

FIG. 10 shows uptake of [⁶⁸Ga]Ga-H₃P₂O₈ in E. coli K12 and heat killed E. coli K12.

FIG. 11 shows a biodistribution of [⁶⁸Ga]Ga-H₃P₂O₈ in normal and thigh infected mice with E. coli at 120 minutes post-infection.

FIG. 12 shows an uptake of [⁶⁸Ga]Ga-H₃P₂O₈ in normal thigh and infected (E. coli) thigh and infected thigh over normal thigh, in thigh infected mouse model at 120 minutes post-infection.

FIG. 13 shows representative micro PET images of uptake of [⁶⁸Ga]Ga-H₃P₂O₈ in thigh infected mice with E. coli and normal mice without infection at different timepoints.

FIG. 14 shows the in vitro effect of iron removal from incubation media on the uptake of [⁶⁸Ga]Ga-H₃P₂O₈ in S. aureus using once chelex pretreated (1X), twice chelex pretreated (2X) and thrice chelex pretreated (3X) incubation medium.

FIG. 15 shows the in vitro uptake of [⁶⁸Ga]Ga-H₃P₂O₈ in thrice (three times) chelex pretreated incubation medium in alive (live) and heat killed S. aureus.

FIG. 16 shows our study design for evaluation of [⁶⁸Ga]Ga-H₃P₂O₈ as an infection imaging PET probe for non-invasive estimation of infection in a chronic foreign body osteomyelitis rat model.

FIG. 17 shows representative PET/CT images of uptake of [⁶⁸Ga]Ga-H₃P₂O₈ in an osteomyelitis foreign body rat model with Methicillin-resistant Staphylococcus aureus (MRSA) infection, a normal rat model, and in foreign body inflammation rat model at different time points post-injection.

FIG. 18 shows a comparison of the SUV of [⁶⁸Ga]Ga-H₃P₂O₈ at different time points post-injection in a normal rat model, osteomyelitis foreign body rat model with Methicillin-resistant Staphylococcus aureus (MRSA) infection, and in inflammation foreign body rat model as determined by PET image analysis.

FIG. 19 shows a comparison of the uptake of [⁶⁸Ga]Ga-H₃P₂O₈ in a normal rat model, osteomyelitis foreign body rat model with Methicillin-resistant Staphylococcus aureus (MRSA) infection, and inflammation foreign body rat model expressed as SUV ratios (normal tibia or infected tibia or inflamed tibia/contralateral tibia) as determined by ex vivo biodistribution.

FIG. 20 shows representative PET/CT images of comparative uptake of [⁶⁸Ga]Ga-H₃P₂O₈ and [⁶⁸Ga]Ga-citrate in an osteomyelitis foreign body rat model with Methicillin-resistant Staphylococcus aureus (MRSA) infection at different time points post-injection.

FIG. 21 shows a comparison of the SUV of [⁶⁸Ga]Ga-H₃P₂O₈ in the osteomyelitis foreign body rat model with Methicillin-resistant Staphylococcus aureus (MRSA) infection and in the normal rat model at different time points post-injection as determined by the PET image analysis.

FIG. 22 shows a comparison of the SUV of [⁶⁸Ga]Ga-H₃P₂O₈ in the osteomyelitis foreign body rat model with Methicillin-resistant Staphylococcus aureus (MRSA) infection and in the inflammation rat model at 120 minutes post-injection as determined by the ex vivo biodistribution.

FIG. 23 shows a comparison of the SUV of [⁶⁸Ga]Ga-H₃P₂O₈ and [⁶⁸Ga]Ga-citrate in Methicillin-resistant Staphylococcus aureus (MRSA) infected tibia, contralateral tibia and muscle at different time points post-injection as determined by PET image analysis.

FIG. 24 shows a comparison of uptake of [⁶⁸Ga]Ga-H₃P₂O₈ and [⁶⁸Ga]Ga-citrate at 120 minutes post-injection in areas of Methicillin-resistant Staphylococcus aureus (MRSA) infection and inflammation expressed as SUV ratios (Operated tibia/contralateral tibia) as determined by the ex vivo biodistribution.

FIG. 25 shows a comparison of uptake of [⁶⁸Ga]Ga-H₃P₂O₈, [⁶⁸Ga]Ga-Citrate and [¹⁸F]FDG in an osteomyelitis foreign body rat model with Methicillin-resistant Staphylococcus aureus (MRSA) using representative PET/CT images at different time points post-injection.

FIG. 26 shows a comparison of the SUV of [⁶⁸Ga]Ga-H₃P₂O₈, [⁶⁸Ga]Ga-citrate and [¹⁸F]FDG in infected tibia in an osteomyelitis foreign body rat model with Methicillin-resistant Staphylococcus aureus (MRSA) infection at different time points post-injection as determined by PET image analysis.

FIG. 27 shows a comparison of the SUV of [⁶⁸Ga]Ga-H₃P₂O₈, [⁶⁸Ga]Ga-citrate and [¹⁸F]FDG in inflamed tibia in foreign body inflammation rat model at different time points post-injection as determined by PET image analysis.

FIG. 28 shows a comparison of uptake of [⁶⁸Ga]Ga-H₃P₂O₈, [⁶⁸Ga]Ga-citrate and [¹⁸F]FDG at 120 minutes post-injection in areas of Methicillin-resistant Staphylococcus aureus (MRSA) infection and inflammation expressed as SUV ratios (Operated tibia/contralateral tibia) as determined by the ex vivo biodistribution.

FIG. 29 shows a comparison of uptake of [⁶⁸Ga]Ga-H₃P₂O₈ in an osteomyelitis foreign body rat model with Methicillin-resistant Staphylococcus aureus (MRSA) or Klebsiella pneumoniae (K. pneumoniae) or Pseudomonas aeruginosa (P. aeruginosa) infection at different time points post-injection.

FIG. 30 shows a comparison of the SUV of [⁶⁸Ga]Ga-H₃P₂O₈ in an osteomyelitis foreign body rat model with Methicillin-resistant Staphylococcus aureus (MRSA) or Klebsiella pneumoniae (K. pneumoniae) or Pseudomonas aeruginosa (P. aeruginosa) infection at different time points post-injection as determined by PET image analysis.

FIG. 31 shows a comparison of uptake of [⁶⁸Ga]Ga-H₃P₂O₈ at 120 minutes post-injection in an osteomyelitis foreign body rat model with Methicillin-resistant Staphylococcus aureus (MRSA) or Klebsiella pneumoniae (K. pneumoniae) or Pseudomonas aeruginosa (P. aeruginosa) infected tibia expressed as SUV ratios (Operated tibia/contralateral tibia) as determined by the ex vivo biodistribution.

FIG. 32 shows a comparison of the SUV of [⁶⁸Ga]Ga-H₃P₂O₈ in infected tibia and contralateral tibia in an osteomyelitis foreign body rat model with Methicillin-resistant Staphylococcus aureus (MRSA) or Klebsiella pneumoniae (K. pneumoniae) or Pseudomonas aeruginosa (P. aeruginosa) infection, inflammation in foreign body inflammation rat and normal tibia in uninfected rat at different time points post-injection as determined by PET image analysis.

FIG. 33 shows a comparison of the SUV of [⁶⁸Ga]Ga-H₃P₂O₈ in infected tibia, contralateral tibia, infected muscle, contralateral muscle in an osteomyelitis foreign body rat model with Methicillin-resistant Staphylococcus aureus (MRSA) or Klebsiella pneumoniae (K. pneumoniae) or Pseudomonas aeruginosa (P. aeruginosa) infection at different time points post-injection as determined by the ex vivo biodistribution at 120 minutes post-injection.

FIG. 34 shows a comparison of the SUV of [⁶⁸Ga]Ga-H₃P₂O₈ in an osteomyelitis foreign body rat model with Klebsiella pneumoniae (K. pneumoniae) infection and in the inflammation rat models as determined by the ex vivo biodistribution at 120 minutes post-injection.

FIG. 35 shows a comparison of the SUV of [⁶⁸Ga]Ga-H₃P₂O₈ in an osteomyelitis foreign body rat model with Pseudomonas aeruginosa (P. aeruginosa) infection and in the inflammation rat models as determined by the ex vivo biodistribution at 120 minutes post-injection.

FIG. 36 shows in vitro uptake of [⁶⁸Ga]Ga-H₃P₂O₈ in different bacterial strains.

### DETAILED DESCRIPTION OF THE INVENTION

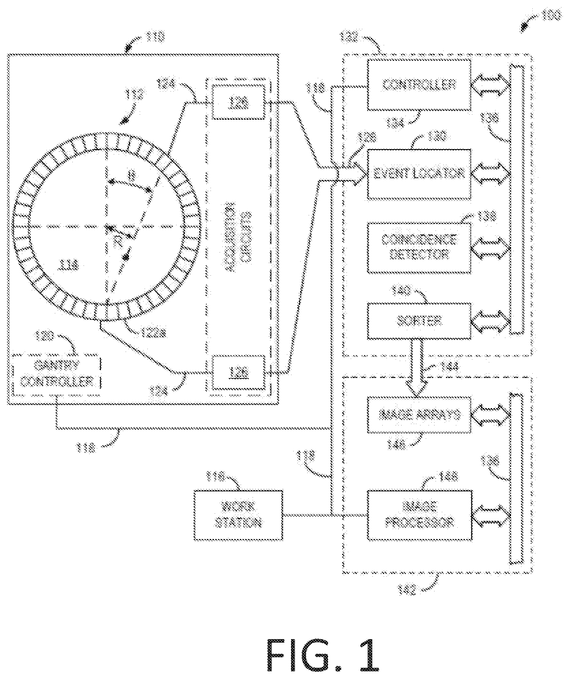

Referring now to FIG. 1, a PET system 100 that can be used with a PET probe of the present invention comprises an imaging hardware system 110 that includes a detector ring assembly 112 about a central axis or bore 114. An operator workstation 116 including a commercially available processor running a commercially available operating system communicates through a communications link 118 with a gantry controller 120 to control operation of the imaging hardware system 110.

The detector ring assembly 112 is formed of a multitude of radiation detector units 122 that produce a signal responsive to detection of a photon on communications line 124 when an event occurs. A set of acquisition circuits 126 receive the signals and produce signals indicating the event coordinates (x, y) and the total energy associated with the photons that caused the event. These signals are sent through a cable 128 to an event locator circuit 130. Each acquisition circuit 126 also produces an event detection pulse that indicates the exact moment the interaction took place. Other systems utilize sophisticated digital electronics that can also obtain this information regarding the precise instant in which the event occurred from the same signals used to obtain energy and event coordinates.

The event locator circuits 130 in some implementations, form part of a data acquisition processing system 132 that periodically samples the signals produced by the acquisition circuits 126. The data acquisition processing system 132 includes a general controller 134 that controls communications on a backplane bus 136 and on the general communications network 118. The event locator circuits 130 assemble the information regarding each valid event into a set of numbers that indicate precisely when the event took place and the position in which the event was detected. This event data packet is conveyed to a coincidence detector 138 that is also part of the data acquisition processing system 132.

The coincidence detector 138 accepts the event data packets from the event locator circuit 130 and determines if any two of them are in coincidence. Coincidence is determined by a number of factors. First, the time markers in each event data packet must be within a predetermined time window, for example, 0.5 nanoseconds or even down to picoseconds. Second, the locations indicated by the two event data packets must lie on a straight line that passes through the field of view in the scanner bore 114. Events that cannot be paired are discarded from consideration by the coincidence detector 138, but coincident event pairs are located and recorded as a coincidence data packet. These coincidence data packets are provided to a sorter 140. The function of the sorter in many traditional PET imaging systems is to receive the coincidence data packets and generate memory addresses from the coincidence data packets for the efficient storage of the coincidence data. In that context, the set of all projection rays that point in the same direction (θ) and pass through the scanner's field of view (FOV) is a complete projection, or “view”. The distance (R) between a particular projection ray and the center of the FOV locates that projection ray within the FOV. The sorter 140 counts all of the events that occur on a given projection ray (R, θ) during the scan by sorting out the coincidence data packets that indicate an event at the two detectors lying on this projection ray. The coincidence counts are organized, for example, as a set of two-dimensional arrays, one for each axial image plane, and each having as one of its dimensions the projection angle θ and the other dimension the distance R. This θ by R map of the measured events is call a histogram or, more commonly, a sinogram array. It is these sinograms that are processed to reconstruct images that indicate the number of events that took place at each image pixel location during the scan. The sorter 140 counts all events occurring along each projection ray (R, θ) and organizes them into an image data array.

The sorter 140 provides image datasets to an image processing/reconstruction system 142, for example, by way of a communications link 144 to be stored in an image array 146. The image arrays 146 hold the respective datasets for access by an image processor 148 that reconstructs images. The image processing/reconstruction system 142 may communicate with and/or be integrated with the work station 116 or other remote work stations.

The PET system 100 provides an example emission tomography system for acquiring a series of medical images of a subject during an imaging process after administering a pharmaceutically acceptable composition including a PET probe as described herein. The system includes a plurality of detectors configured to be arranged about the subject to acquire gamma rays emitted from the subject over a time period relative to an administration of the composition to the subject and communicate signals corresponding to acquired gamma rays. The system also includes a reconstruction system configured to receive the signals and reconstruct therefrom a series of medical images of the subject. In one version of the system, a second series of medical images is concurrently acquired using an x-ray computed tomography imaging device. In one version of the system, a second series of medical images is concurrently acquired using a magnetic resonance imaging device.

Administration to the subject of a pharmaceutical composition including a PET probe of the invention can be accomplished intravenously, intraarterially, intrathecally, intramuscularly, intradermally, subcutaneously, intraperitonially or intracavitary. A “subject” is a mammal, preferably a human. In the method of the invention, sufficient time is allowed after administration of a detectable amount of the PET probe of the invention such that the PET probe can accumulate in a target region of the subject. A “detectable amount” means that the amount of the PET probe that is administered is sufficient to enable detection of accumulation of the PET probe in a subject by a medical imaging technique.

One non-limiting example method of imaging according to the invention involves the use of an intravenous injectable composition including a PET probe of the invention. A positron emitting atom of the PET probe gives off a positron, which subsequently annihilates and gives off coincident gamma radiation. This high energy gamma radiation is detectable outside the body using positron emission tomography imaging, or positron emission tomography concurrent with computed tomography imaging (PET/CT), or positron emission tomography with magnetic resonance imaging (PET/MRI). With PET/CT, the location of the injected and subsequently accumulated PET probe within the body can be identified.

The PET image results can be reported in terms of a standardized uptake value, SUV, which is the ratio of the image-derived radioactivity concentration c_{img} and the whole body concentration of the injected radioactivity cᵢₙⱼ, or c_{img}/cᵢₙⱼ. The c_{img} data may be the pixel intensities of a calibrated PET image. Calculated SUV data can then be visualized as parametric SUV image. Alternatively, groups of such pixels may be selected e.g., by manually drawing or otherwise segmenting a region of interest (ROI) on the PET image. Then average intensity of the ROI may be used as c_{img} input to calculate SUV values.

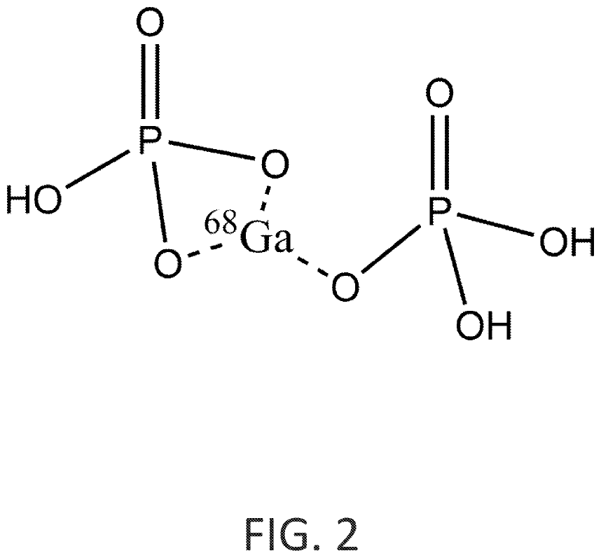

In one aspect, the present disclosure provides a composition comprising: a radiopharmaceutical including: (i) cations comprising a positron emitter, and (ii) anions selected from the group consisting of [PO₄]³⁻, [HPO₄]²⁻, [H₂PO₄]⁻, and mixtures thereof, wherein the cations are ionically bonded to the anions. The positron emitter can be selected from the group consisting of ¹¹C, ¹³N, ¹⁵O, ¹⁸F, ^{34m}Cl, ³⁸K, ⁴³Sc, ⁴⁴Sc, ⁴⁵Ti, ⁵¹Mn, ⁵²Mn, ^{52m}Mn ⁵²Fe, ⁵³Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ^{189/189m}Hg, ¹⁹¹Hg, ^{191m}Hg, ¹⁹³Hg, ^{193m}Hg, ¹⁹⁵Hg, ^{195m}Hg, ⁷⁵Br, ⁷⁶Br, ⁸²Rb, ⁸⁶Y, ⁸⁹Zr, ⁹⁰Nb, ^{94m}Tc, ^{110m}In, ¹¹⁵Sb, ^{116m2}Sb, ^{118m2}Sb, ¹¹⁸Sb, ¹²⁰Sb, and ¹²⁴I. The positron emitter can be ⁶⁸Ga. The radiopharmaceutical can have the formula: [⁶⁸Ga]Ga-H₃P₂O₈. A ⁶⁸Ga³⁺ ion can be ionically associated with a [H₂PO₄]¹⁻ ion and a [HPO₄]²⁻ ion. In one example, the radiopharmaceutical can have the following structure, also shown in FIG. 2:

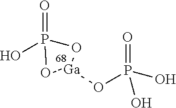

Additionally, and alternatively, the radiopharmaceutical can have the formula: [⁶⁷Ga]Ga-H₃P₂O₈. Besides the radiopharmaceutical, the composition may include excipients, adjuvants, additional radiopharmaceuticals, drugs, and any combination thereof. An image can be acquired using the radiopharmaceutical and a medical imaging technique selected from positron emission tomography imaging, positron emission tomography with computed tomography imaging, positron emission tomography with magnetic resonance imaging, and positron emission tomography with single-photon emission computed tomography. In the case where the radiopharmaceutical is [⁶⁷Ga]Ga-H₃P₂O₈, an image may be acquired using single-photon emission computed tomography, or single-photon emission computed tomography with magnetic resonance imaging.

In another aspect, the present disclosure provides a method for in vivo imaging of a subject. The method can comprise: (a) administering to the subject the composition; (b) waiting a time sufficient to allow the radiopharmaceutical to accumulate at a tissue or cell site or an organ of interest to be imaged; and (c) imaging the cells or tissues or the organ of interest with a medical imaging technique.

The composition can be administered by injection (parenteral administration) including subcutaneous administration, intramuscular administration, intravenous administration, and intrathecal administration.

A time sufficient to allow the radiopharmaceutical to accumulate at a tissue or cell site or an organ of interest to be imaged can vary. For a Ga-68 labeled version as a PET probe, the time can be about 15 minutes, about 30 minutes, about 45 minutes, about 60 minutes, about 75 minutes, about 90 minutes, about 105 minutes, about 120 minutes, about 135 minutes, about 150 minutes, about 165 minutes, or about 180 minutes. For other radiolabeling isotopes, the time can be anywhere between 15 minutes to 3 days. In one example, the medical imaging technique can be positron emission tomography.

In another aspect, the present disclosure provides a method of imaging a subject by positron emission tomography. The method can comprise: (a) administering the composition to the subject; (b) using a plurality of detectors to detect gamma rays emitted from the subject and to communicate signals corresponding to the detected gamma rays; and (c) reconstructing from the signals a series of medical images of a region of interest of the subject.

In another aspect, the present disclosure provides an imaging method. The method can comprise acquiring an image of a subject to whom a detectable amount of the radiopharmaceutical of the composition has been administered.

In another aspect, the present disclosure provides a method for detecting bacterial infection in a subject. The method can comprise: (a) administering to the subject the composition; (b) waiting a time sufficient to allow the radiopharmaceutical to accumulate at a tissue or cell site or an organ of interest to be imaged; and (c) imaging the cells or tissues or the organ of interest with a medical imaging technique. The radiopharmaceutical is adapted for targeting a site of one of both of gram-negative and gram-positive bacterial infection of any cell or tissues or organs. The bacterial infection can be resultant from implantation, orthopedic surgery, or trauma. The bacterial infection can be osteomyelitis, septic arthritis or a prosthetic joint infection. The bacterial infection can affect the lungs, heart, stomach, spleen, bone, brain, gut, liver, kidneys, adipose tissue, cecum, eye, bladder, intestines, or muscle tissue. The bacterial infection can be at the site of a vascular graft, interventional graft, or a foreign body surgical implant. In one example, the site of bacterial infection can be a bone or graft. In another example, the site of bacterial infection can be adjacent a bone or adjacent a graft. In another example, the bacterial infection can be related to an orthopedic surgical implant at a femur, thigh bone, shoulder bone, arm bone, wrist bone, or hand bone. In other examples, the site of bacterial infection can be a surgical implant, for example a spine infusion, a hip replacement, or a knee replacement. In other examples, the site of infection can be the heart, adjacent the heart, or a cardiac device such as a stent or pacemaker. In some examples, the site of bacterial infection can be a myocardial infection.

Bacterial strains causing the infection can include Staphylococcus, Streptococcus, Pseudomonas or combinations thereof. In some examples, the infection can be caused by Staphylococcus aureus, Staphylococcus epidermidis, Pseudomonas aeruginosa, Klebsiella pneumoniae, or Escherichia coli. In certain examples, the infection can be caused by methicillin-resistant S. aureus (MRSA).

In a certain aspect, the present disclosure provides a method for identifying a Methicillin-resistant Staphylococcus aureus bacterial infection in a subject. The method may comprise: (a) administering to a subject the composition wherein the radiopharmaceutical is taken up at an infection in a tissue or cell site or an organ of interest in the subject; and (b) acquiring an image of the cells or tissues or the organ of interest to detect the presence or absence of the Methicillin-resistant Staphylococcus aureus bacterial infection in the subject.

In another aspect, the present disclosure provides a method for detecting or ruling out a condition involving a bacterial infection in a subject. The method may comprise: (a) administering to a subject the composition wherein the radiopharmaceutical is targeted to a bacterial infection at a tissue or cell site or an organ of interest in the subject; and (b) acquiring an image of the cells or tissues or the organ of interest to detect the presence or absence of a bacterial infection in the subject. In some aspects, the pharmaceutical accumulates in the tissue or cell site or organ of interest, which are included in a region of interest, thereby increasing the SUV of the region of interest, and thereby detecting the presence of a bacterial infection in the tissue or cell site or organ of interest. In some aspects, the pharmaceutical accumulates near or around the tissue or cell site or organ of interest, increasing the SUV near or around the region of interest, and thereby detecting the presence of a bacterial infection near or around the tissue or cell site or organ of interest. In other aspects, the pharmaceutical does not accumulate in the tissue or cell site or organ of interest, and the SUV does not increase in the region of interest, thereby ruling out a bacterial infection in the tissue or cell site or organ of interest.

In another aspect, the present disclosure provides a method for discerning the strain of bacteria causing a bacterial infection in a subject. The method may comprise: (a) administering to a subject the composition wherein the radiopharmaceutical is targeted to a bacterial infection at a tissue or cell site or an organ of interest in the subject; (b) acquiring an image of the cells or tissues or the organ of interest. The method may further comprise (c) comparing the SUV of the region of interest to a predetermined value to determine whether the SUV is greater than or less than the predetermined value, wherein the predetermined value is indicative of a specific bacterial strain. For example, if the bacterial infection is due to MRSA, the SUV at 120 min may be greater than 2, greater than 2.5, or greater than 3. In another example, if the bacterial infection is due to K. pneumoniae or P. aeruginosa, the SUV at 120 min may be greater than 1, greater than 1.2, or greater than 1.5.

In another aspect, the present disclosure provides a method for detecting or ruling out a condition involving inflammation in a subject. The method may comprise: (a) administering to a subject the composition of the present disclosure wherein the radiopharmaceutical is targeted to a site of inflammation in a tissue or cell site or an organ of interest in the subject; and (b) acquiring an image of the cells or tissues or the organ of interest to detect the presence or absence of an inflammation. The method may further comprise (c) comparing the data from the image of the cells or tissues or the organ of interest to a previously acquired data from an image of a known inflammation or a known infection.

The inflammation can be chronic inflammation, neurological inflammation, or inflammation in the extremities of the subject.

### EXAMPLES

The following Examples are provided to demonstrate and further illustrate certain embodiments and aspects of the present invention and are not to be construed as limiting the scope of the invention. The statements provided in the Example are presented without being bound by theory.

### Example 1

### Overview of Example 1

Current methods of non-invasively imaging infections (X-rays, CT, MRI) are only usable when infections have caused morphological changes to organs, and current PET imaging techniques produce blurry images and are non-specific. An accurate and non-invasive imaging technique could be used to detect infections following a surgical procedure. For example, infection can occur following hip replacements, implantation of cardiac devices, hip, knee or other joint replacement, implants for mending broken bones, and interventional and vascular grafting.

In Example 1, we investigated a novel chemical/molecular entity labeled with a positron emission tomography (PET) isotope ⁶⁸Ga, which selectively identifies a site of bacterial infection in the host body. As of now, there is no direct and economical imaging method available which can noninvasively, selectively, and specifically identify bacteria in their early or chronic state at the site of infection. No existing non-invasive imaging methods, including X-ray, computed tomography (CT), ultrasound and magnetic resonance imaging (MRI), directly visualizes bacteria; instead, they depend on recognition of effects of host response to the pathogen, and are only able to detect significant morphological change in the infected organ or tissue. A similar limitation exists with [¹¹¹In]In-WBC imaging where the body's defenses against infection are used as a surrogate marker. We developed a PET probe, when injected intravenously, that directly images bacteria at the site of infection. The developed PET probe has been preliminarily evaluated in a murine thigh infection model and an osteomyelitis foreign body model using E. coli, and S. aureus, as infecting agents, respectively. The developed PET probe has also been evaluated in orthopedic surgical inflammation and in normal rodents.

In this Example 1, we evaluated the use of the novel PET probe [⁶⁸Ga]Ga-H₃P₂O₈ in a rat model of foreign-body osteomyelitis. We evaluated and compared the standard uptake value (SUV) of the PET probe [⁶⁸Ga]Ga-H₃P₂O₈ around infected implants and non-infected implants to differentiate between inflammation and bacterial infection. Given the structural simplicity, ease of synthesis, and low cost of manufacturing, the developed PET probe [⁶⁸Ga]Ga-H₃P₂O₈ holds high commercial and clinical value as a noninvasive PET imaging tool to diagnose bacterial infections such as foreign body infections complicating in orthopedic surgeries involving implants, where inflammation is a non-specific finding that limits use of other imaging modalities. Inflammation is part of surgery. Infection can hide for years, then spread quickly in weeks. PET probes radiolabeled with ⁶⁸Ga have been used, such as Ga-68-citrate. However, our compound [⁶⁸Ga]Ga-H₃P₂O₈ is a new molecule with a distinct formulation than any previously reported PET probes and can be manufactured (Example 1) in less than ten minutes.

### Scope of Example 1

With advancements in medicine, there has been a significant rise in surgical procedures involving implantation of prosthetic devices with a consequent increase in the number of microbial infections associated with these surgical procedures/devices. The incidence of infection in orthopedic trauma patients is high and can range from 5-10% depending upon type of injury, location, and severity of trauma [Ref. 1]. Significant increases have been observed in hip, knee and shoulder replacement procedures since 2000-2010, with 1.39 million hip replacements in the year 2000, increased to 3.1 million in the year 2010, among inpatients aged 45 and over [Ref. 2]. In addition to hip replacements, other surgical procedures such as implantation of cardiac devices, instrumented spinal fusions, and vascular grafts can be associated with serious microbial infections [Ref. 3]. However, after invasive surgical procedures, it can be difficult to differentiate between inflammation and infection in these patients, thereby affecting the course of the treatment. Furthermore, other medical conditions like osteomyelitis, diabetic foot infection, endocarditis, and abdominopelvic infection, that require definitive diagnosis for appropriate management. In addition, bacterial biofilms are a concern in the clinical community, because in bacterial biofilms, bacteria adhere to a self-produced matrix and remain hidden from the host's immune response, and from the effect of antibiotics or antimicrobial treatment [Ref. 4-5]. In fact, biofilm is considered as a common reason for recalcitrant chronic infection. These developments have created an unmet clinical need for a reliable, sensitive, and specific noninvasive imaging tool to identify bacterial infection and differentiate it from inflammation. In fact, bacterial infections are considered one of the main causes of the patient mortality and morbidity [Ref. 6]. Diagnosis of bacterial infection can be challenging especially in the early stage and in deep seated infections. Normally bacterial infections are characterized by symptoms, signs and eventually by isolating the pathogens [Ref. 6]. Mainstream non-invasive imaging techniques used in diagnosis of bacterial infections are X-ray, CT, ultrasound, white blood cell (WBC) scans, and MRI, but these techniques are only effective when pathogen has caused significant morphological change in the infected organ or tissue, findings which can be non-specific.

Various mechanisms related to infection have been explored for the development of an imaging probe including imaging of increased levels of hydroxyapatite using ^{99m}Tc labeled medronic acid also called [^{99m}Tc]Tc-MDP but found to be nonspecific, direct imaging of radiolabeled white blood cells (WBCs) using [¹¹¹In]In-WBC and [^{99m}Tc]Tc-WBC but both needed extraction of WBCs followed by radiolabeling and reinjection. Moreover, WBC based imaging requires at least 4,000 WBCs for effective labeling and imaging, which can be challenging to achieve. Images have relatively poor resolution with an [¹¹¹In]In-WBC probe. Metabolic trapping of ¹⁸F labeled glucose as [¹⁸F]Fluorodeoxyglucose ([¹⁸F]FDG), sorbital as [¹⁶F]Fluorodeoxysorbital ([¹⁸F]FDS), maltose as [¹⁸F]Fluoromaltose and [¹⁸F]Fluoromaltotriose have been explored but found to be nonspecific as well [Ref. 3,6-9]. Another way to image or identify the infection is based on targeting the bacterial needs; iron (Fe) is an essential element for both humans and bacteria [Ref. 10]. There are two ways bacteria acquire Fe: (1) direct intake through a membrane bound transferrin receptor [Ref. 10-11]; and (2) through secretion and reabsorption of siderophores after Fe chelation. Siderophores are Fe chelating molecules secreted by the bacteria to acquire Fe. Bacteria rely on both membrane-bound transferrin receptors and siderophores to acquire Fe for their growth, whereas the host or human body employs its iron binding proteins like lactoferrin, transferrin and ferritin to sequester or remove Fe from the site of infection. This adaptation for survival results in competition between pathogens and the host for the Fe [Ref. 10]. Due to the importance of the Fe in the bacterial life cycle, Fe targeted imaging has great potential to identify bacterial location in the host. Recently, various siderophores including desferioxamine [Ref. 12], citrate [Ref. 13], pyoverdine [Ref. 14], and enterobactin and enterobactin analogs [Ref. 15]were radiolabeled with either ^{67/68}Ga or ⁸⁹Zr isotopes and evaluated in preclinical animal models for infection imaging potential with mixed results. SPECT agent [⁶⁷Ga]Ga-Citrate is used clinically, and has been clinically studied [Ref. 16].

Due to similar size of gallium (Ga³⁺ ionic radius is 76 picometers) as compared to iron (Fe³⁺ ionic radius can be between 69-79 picometers), it is believed that ⁶⁸Ga may serve as a surrogate marker for Fe and be taken up by bacteria as they take up Fe using both membrane-bound transferrin receptor and siderophores. In addition, it is expected that ⁶⁸Ga will conjugate with ferritin, transferrin and lactoferrin and accumulate at the site of bacterial infection. Therefore, in this invention, in vitro and in vivo evaluation of [⁶⁸Ga]Ga-H₃P₂O₈ as a PET probe for infection imaging was performed.

The present Example 1, [⁶⁸Ga]Ga-H₃P₂O₈ has been developed and evaluated as a novel PET imaging probe for noninvasive infection imaging, imaging of infection at the site of orthopedic surgical implants, inflammation imaging, and also their head-to-head comparison.

Method of [⁶⁸Ga]Ga-H₃P₂O₈ Preparation

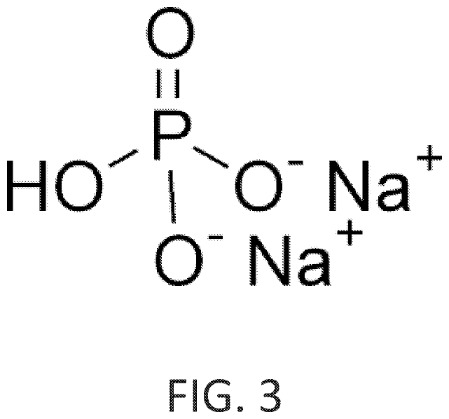

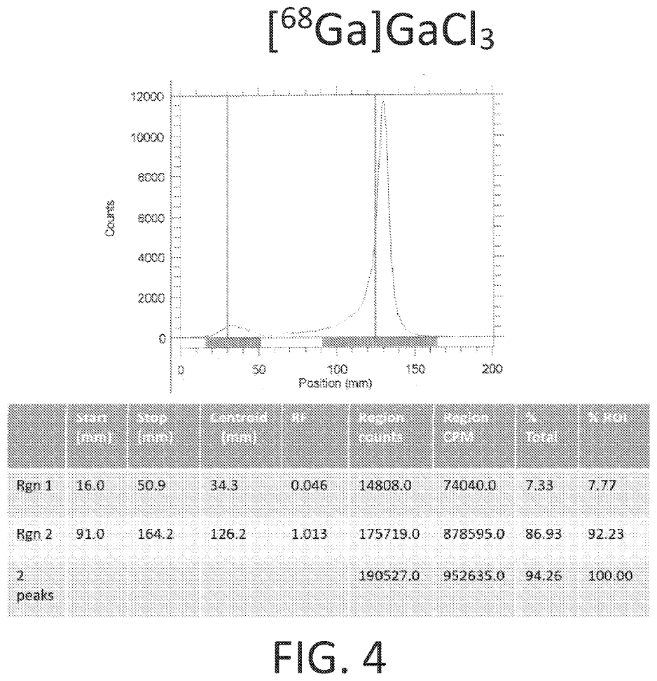

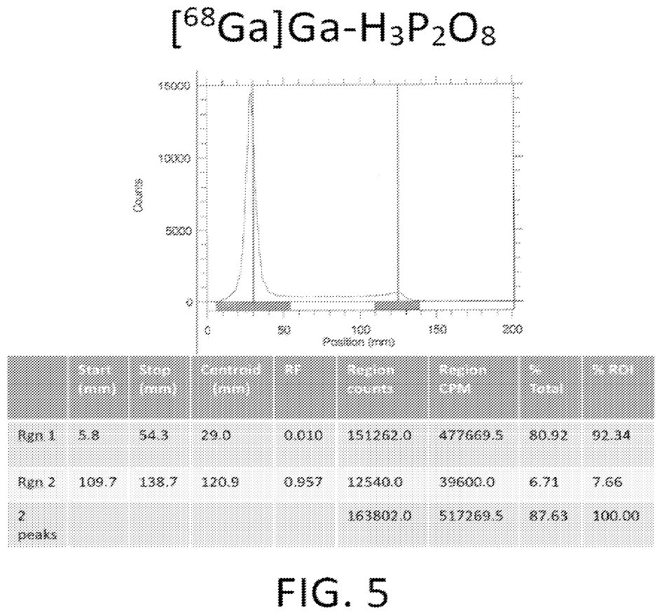

Synthesis of [⁶⁸Ga]Ga-H₃P₂O₈ (FIG. 2) was achieved by mixing [⁶⁸Ga]GaCl₃ (1.0 mL), either eluted from a ⁶⁸Ge/⁶⁸Ga generator or produced by a cyclotron, with 0.8 mL of phosphate buffered saline (PBS, 30×) (see FIG. 3), and stirring the reaction mixture for 5 minutes. After completion of reaction, the pH of the reaction mixture was examined using a pH strip (pH 6.0). The formation of [⁶⁸Ga]Ga-H₃P₂O₈ was confirmed by rad-TLC analysis using 0.0025M ethylene diamine tetraacetic acid disodium salt dihydrate (EDTA disodium, pH 5.0-5.5) as a mobile phase and i-TLC (silica gel coated paper TLC) as a solid phase. The free [⁶⁸Ga]GaCl₃ moved to the solvent front (˜1.0 Rf) (see FIG. 4) and the labeled [⁶⁸Ga]Ga-H₃P₂O₈ stayed at the origin (˜0.0Rf) of the rad-TLC plate (see FIG. 5). After confirmation of the product formation, a resultant solution was filtered through a 0.22 μm sterile filter before being injecting into animal models.

The 30× phosphate buffered saline (PBS, 30×) solution was prepared by dissolving one tablet of PBX (˜1.88 g) in 6.67 mL of deionized water, yielding 0.3 M phosphate buffer, 0.081 M potassium chloride, and 4.11 M sodium chloride, pH 7.4, at 25° C. as a stock solution.

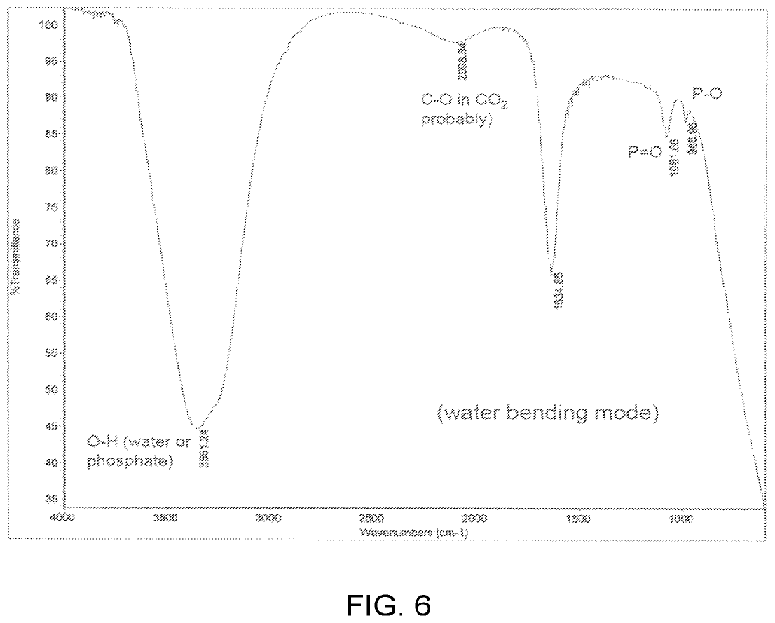

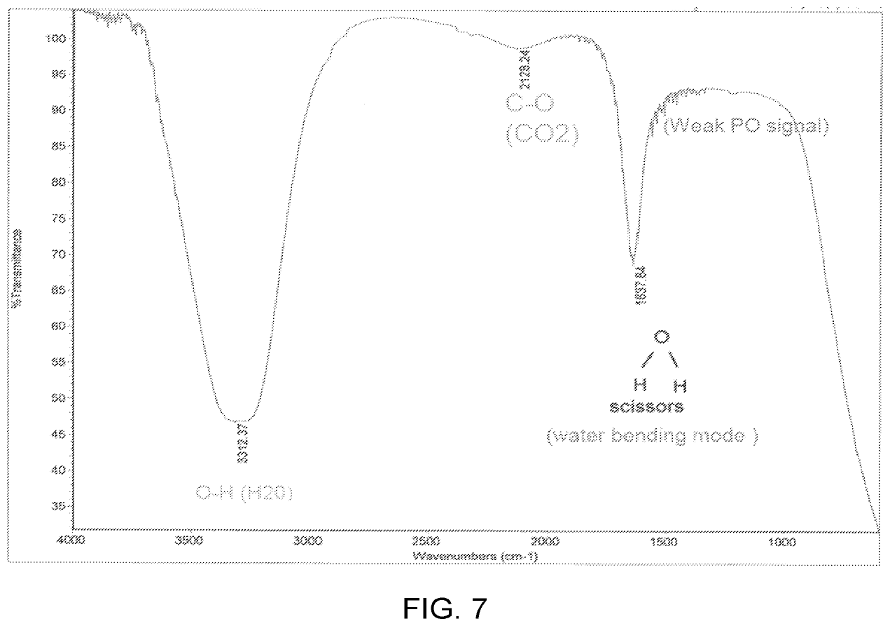

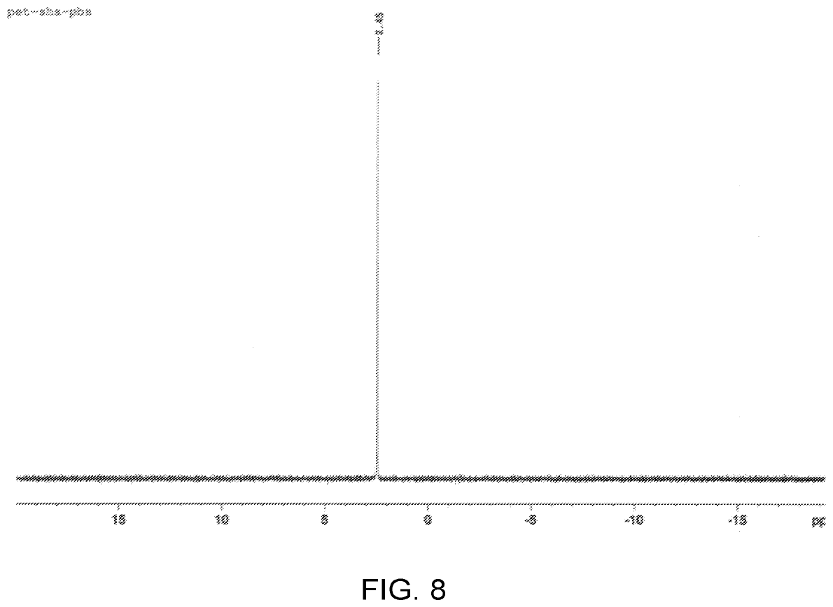

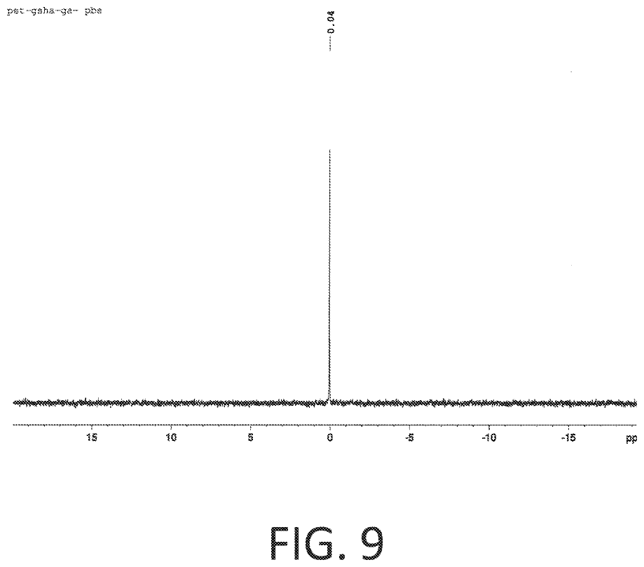

Since radiosynthesis of [⁶⁸Ga]Ga-H₃P₂O₈ was achieved using stock solution of phosphate buffered saline (PBS, 30×), therefore, the final formulation of [⁶⁸Ga]Ga-H₃P₂O₈ also contains phosphate buffer (0.133-0.02M), potassium chloride (0.036-0.005) and sodium chloride (1.81-0.27M), pH 6.0, at 25° C. The molarity of phosphate buffer, potassium chloride and sodium chloride in final formulation of [⁶⁸Ga]Ga-H₃P₂O₈ may vary depending upon the final volume of radiolabel product for animal or human use. IR spectroscopy was performed of PBS in water (see FIG. 6) and Ga-H₃P₂O₈ (see FIG. 7) to confirm formation of [⁶⁸Ga]Ga-H₃P₂O₈. Additionally, formation of [⁶⁸Ga]Ga-H₃P₂O₈ was confirmed via ³¹P-NMR analysis of both phosphate (see FIG. 8) and Ga-H₃P₂O₈ (see FIG. 9) through visualization of an up-field shift in the spectrum.

A stock solution of 0.1M ethylene diamine tetraacetic acid disodium salt dihydrate (EDTA disodium) was prepared and adjusted for required pH using 0.1 M sodium carbonate. Various concentrations of EDTA solution were prepared with variable pH via dilution of stock solution with deionized water and pH adjustment with 0.1M sodium carbonate to find an appropriate mobile phase, which could differentiate between [⁶⁸Ga]Ga-H₃P₂O₈ and [⁶⁸Ga]GaCl₃.

The appropriate TLC mobile phase for the analysis of [⁶⁸Ga]Ga-H₃P₂O₈ from free [⁶⁸Ga]GaCl₃ is 0.0025 M EDTA solution of pH 5.2 (see FIGS. 4-5).

### Data

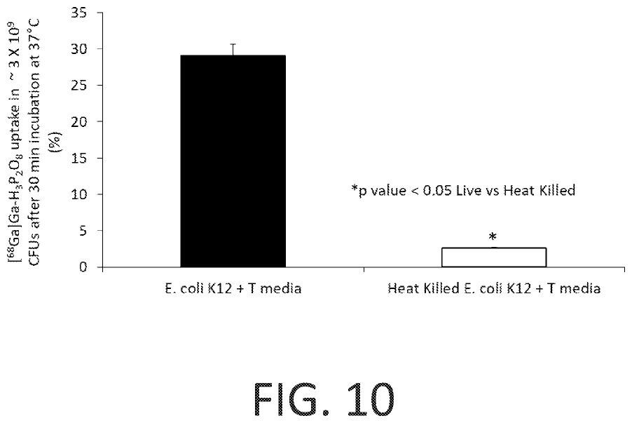

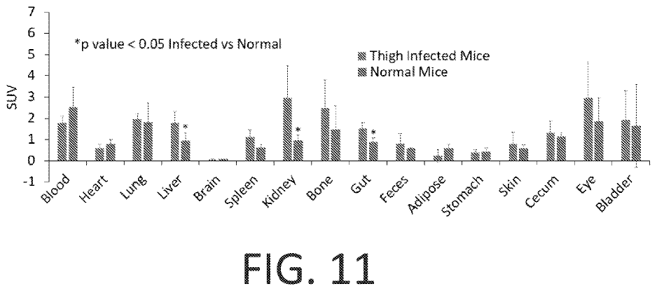

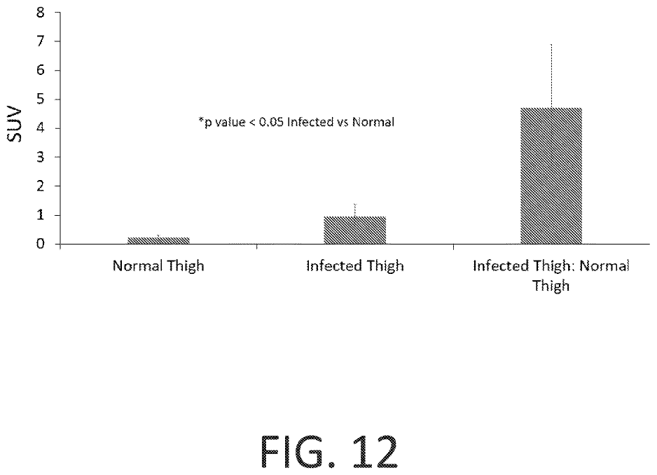

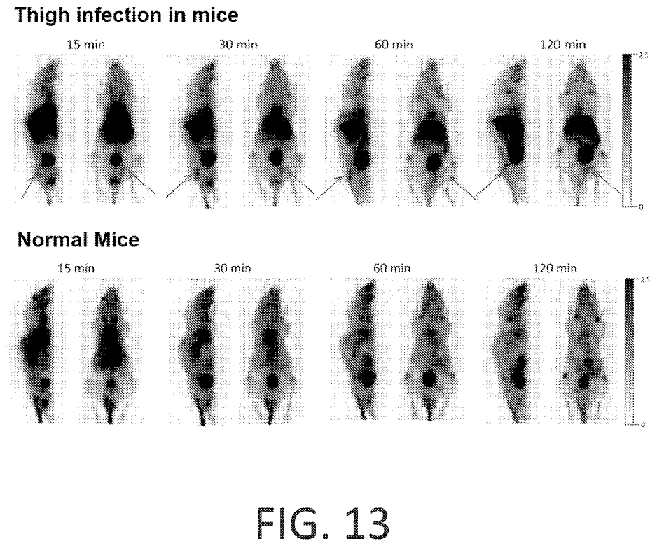

The feasibility of the developed PET probe [⁶⁸Ga]Ga-H₃P₂O₈ in a mouse model of thigh infection as well as uptake in E. coli has been evaluated. The in vitro uptake was performed in living Escherichia coli (gram-negative bacteria) in comparison with heat killed Escherichia coli to demonstrate that uptake of the PET probe [⁶⁸Ga]Ga-H₃P₂O₈ is part of an active mechanism in the living bacteria (see FIG. 10). The biodistribution of developed PET probe in normal and in thigh infected mice has been assessed (see FIG. 11). The results showed significant higher uptake of [⁶⁸Ga]Ga-H₃P₂O₈ in infected thigh over normal thigh. Infected thigh showed 4-fold higher uptake of the developed PET probe over non-infected (normal) thigh in the same mice (see FIG. 12). Micro-PET imaging of normal and thigh-infected mice has also been performed. The site of bacterial infection was evident on micro-PET images (see FIG. 13) with arrows in contrast to the non-infected group of mice. The bacterial site of infection on micro-PET images was confirmed by bacterial culture performed on the biopsied sample. Micro-PET imaging was performed at different time intervals to optimize imaging time. It was found that the site of infection shows uptake of the developed PET probe at 15 minutes post injection and accumulates more probe with better image resolution at 120 minutes post injection (see FIG. 13 and Table 1).

TABLE 1
Biodistribution [⁶⁸Ga]Ga—H₃P₂O₈ in Normal and Different
Infected Mice Models at 120 min Post Injection

| | | Thigh Infected | Lung infected | Lung infected |
|---|---|---|---|---|
| | | Mice^{¥} (SUV ± | Mices^{§} (SUV ± | Mice [symbol] |
| | Normal Mice | STD, n = 4) | STD, n = 4) | (SUV ± STD, |
| | (SUV ± STD) | 2 h post | 24 h post | n = 3) 2 h post |
| Tissues/Organ | N = 5 | infection | infection | infection |
| Blood | 2.76 ± 0.97 | 1.83 ± 0.20 | 2.75 ± 0.39 | 2.09 ± 1.31 |
| Heart | 0.87 ± 0.30 | 0.61 ± 0.14 | 1.04 ± 0.28 | 0.73 ± 0.07 |
| Lung-1 | 2.17 ± 0.85 | 2.13 ± 0.54 | 1.52 ± 0.29 | 2.98 ± 1.02 |
| Lung-2 | 3.22 ± 2.29 | 3.03 ± 0.53 | 1.46 ± 0.51 | 2.56 ± 1.82 |
| Liver | 1.05 ± 0.42 | 2.02 ± 0.89 | 1.89 ± 0.47* | 0.86 ± 0.08 |
| Brain | 0.07 ± 0.02 | 0.81 ± 1.5 | 0.11 ± 0.02* | 0.05 ± 0.01* |
| Spleen | 0.70 ± 0.16 | 0.80 ± 0.69 | 1.75 ± 0.56* | 0.73 ± 0.03 |
| Kidney-1 | 1.11 ± 0.29 | 3.24 ± 1.19* | 1.69 ± 0.66* | 0.92 ± 0.24 |
| Kidney-2 | 0.71 ± 0.20 | 3.28 ± 2.10 | 1.44 ± 0.37* | 1.10 ± 0.09* |
| Bone | 1.88 ± 1.26 | 2.73 ± 1.12 | 2.65 ± 1.75 | 2.70 ± 0.43 |
| Gut | 1.15 ± 0.29 | 1.78 ± 0.10* | 1.72 ± 0.31* | 1.93 ± 0.40 |
| Feces | 0.82 ± 0.28 | 0.93 ± 0.43 | 0.71 ± 0.24 | 1.39 ± 1.10 |
| Adipose | 0.76 ± 0.38 | 0.34 ± 0.34 | 1.13 ± 0.34 | 0.85 ± 0.64 |
| Stomach | 0.49 ± 0.24 | 0.48 ± 0.14 | 0.34 ± 0.13 | 0.46 ± 0.07 |
| Skin | 0.71 ± 0.38 | 0.88 ± 0.63 | 1.08 ± 0.42 | 1.46 ± 0.41 |
| Infected Thigh | — | 1.12 ± 0.43* | — | — |
| Cecum | 1.33 ± 0.60 | 1.74 ± 0.55 | 1.59 ± 0.35 | 1.61 ± 1.29 |
| Eye | 2.30 ± 1.41 | 3.86 ± 1.71 | 1.39 ± 1.17 | 1.42 ± 1.91 |
| Bladder | 2.41 ± 2.84 | 3.11 ± 3.0 | 4.04 ± 5.22 | 3.11 ± 2.62 |
| Normal Thigh | 0.52 ± 0.23 | 0.25 ± 0.05 | 0.33 ± 0.16 | 0.31 ± 0.18 |

*P value < 0.05, ^{¥}studies were performed 2 h post infection with E coli; ^{§}24 h post infection and [symbol] 2 h post infection with E coli.

### Study Design and Approach, Including Methodology and Clinical Impact

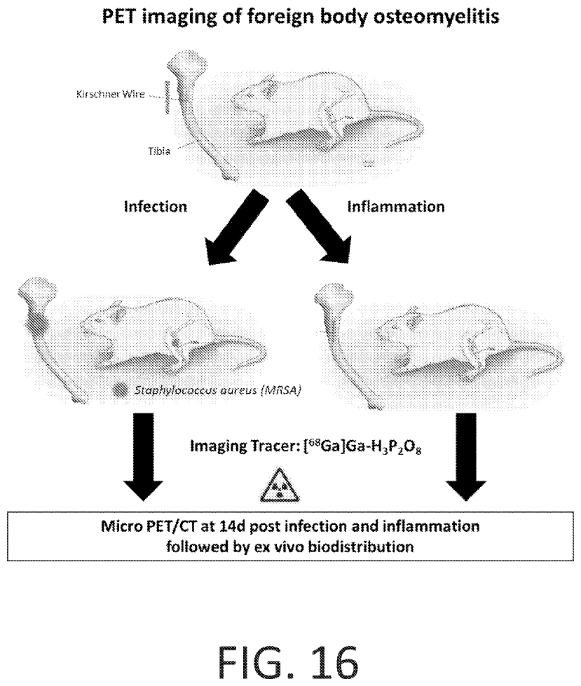

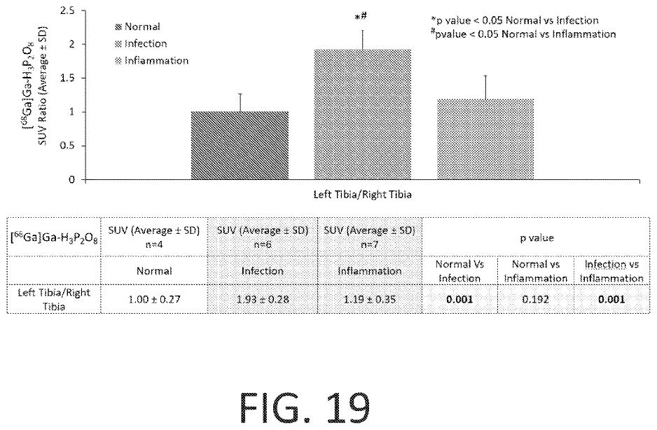

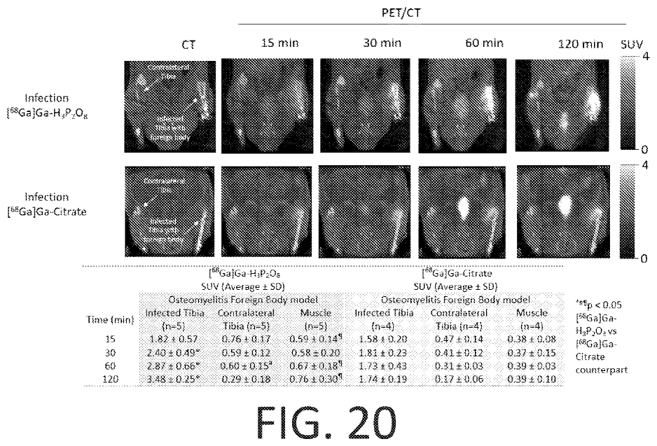

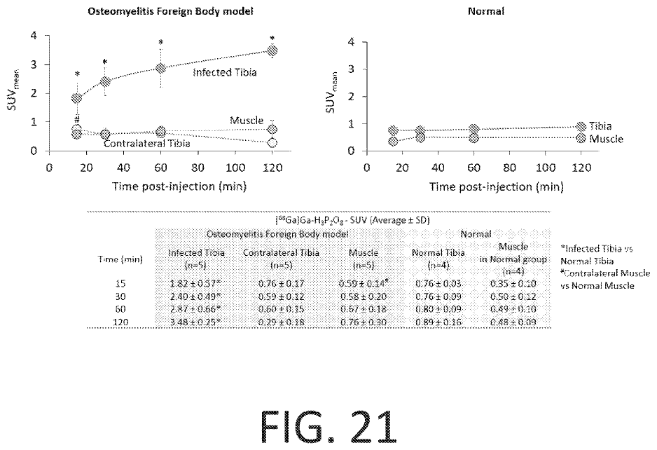

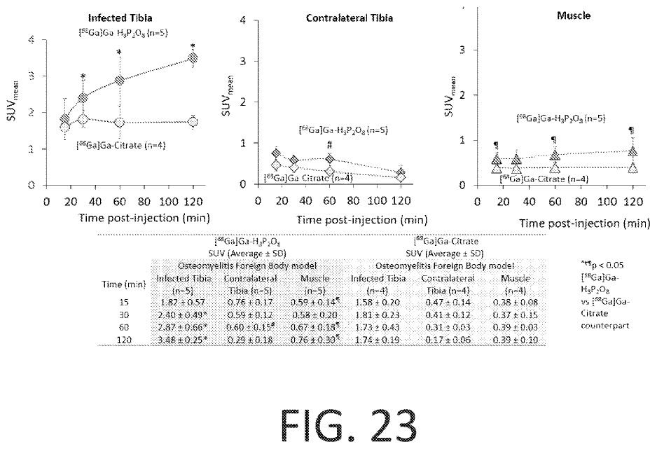

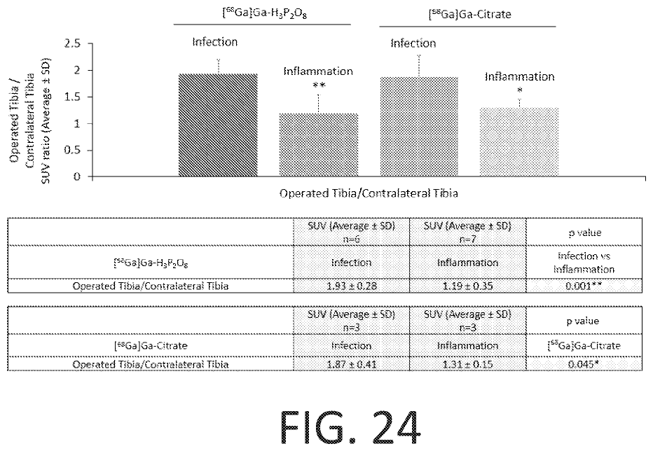

To achieve the objectives of the present invention in this Example, a series of experiments has been designed to evaluate the feasibility of [⁶⁸Ga]Ga-H₃P₂O₈ as a PET infection imaging probe for imaging of infection in orthopedic surgical implants in conjuncture with inherent inflammation caused due to the surgery. It has been established that one of the most common infectious agents in orthopedic surgical implants is Staphylococcus aureus (S. aureus, a gram-positive bacteria), and validated in a rat model of foreign body osteomyelitis (see FIG. 16-24). It has been established that [⁶⁸Ga]Ga-H₃P₂O₈ is highly effective to identify the presence of S. aureus in infections associated with orthopedic surgical implants via PET imaging.

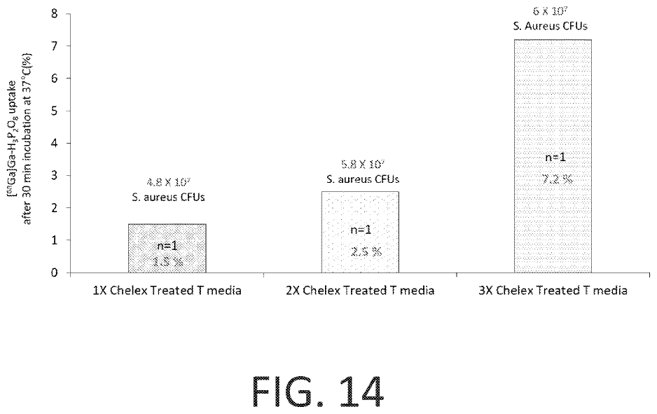

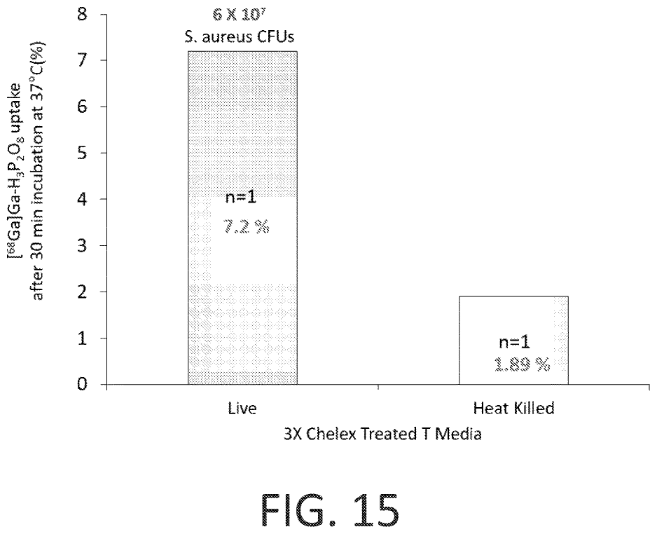

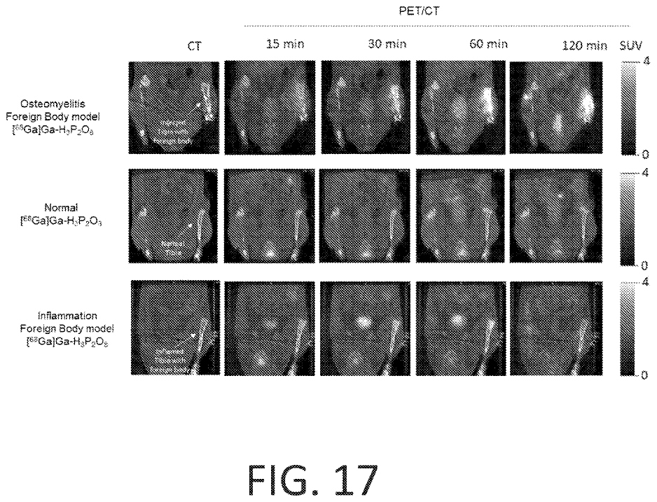

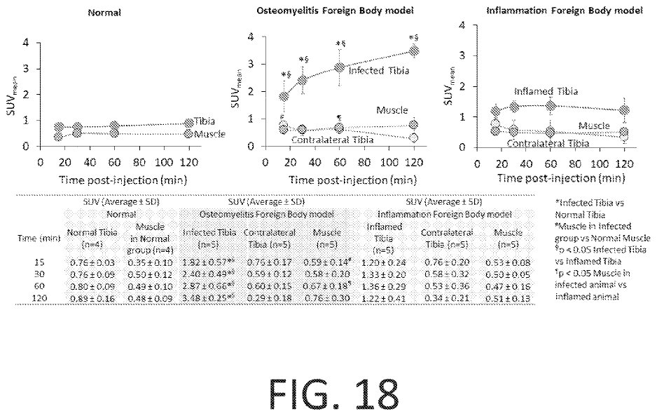

In this Example, in vitro uptake of [⁶⁸Ga]Ga-H₃P₂O₈ was first examined in S. aureus with and without removal of Fe from the media to show that our developed PET probe is indeed a surrogate for Fe (see FIG. 14-15). The PET probe [⁶⁸Ga]Ga-H₃P₂O₈ was evaluated in methicillin-resistant S. aureus (MRSA) foreign-body osteomyelitis in the left tibia with a stainless steel Kirschner wire (K-wire) implant in male Wister rats, following a previously established protocol [Ref. 17]. Briefly, a 1 cm incision was made on the medial tibia and the bone exposed. A 1.5 mm hole was drilled, 0.01 mL of arachidonic acid (a sclerosing agent) and 0.05 mL of a suspension containing MRSA DRL-6169 at 10⁸ cfu/mL was injected into the bone. A 5 mm threaded stainless-steel K-wire was inserted. The hole was closed with dental gypsum and the site closed. After implantation, the muscle and fascia were closed with 3-0 vicryl using simple interrupted sutures. The skin was closed with sterile wound clips and VetBond. The wound was sprayed with Aluspray and Chew Guard. After 7 days of implantation of wire, the animal was put on iron-deficient diet for 7 days and on the day of imaging, the animals were fasted for 4 hours prior to the injection of the PET probe. [⁶⁸Ga]Ga-H₃P₂O₈ (32.81±6.28 MBq, n=18) were injected into the rat model via tail vein injection. The rat model was either a normal rat model or an osteomyelitis foreign body rat model or an inflamed foreign body rat model. PET/CT images were acquired at 15 minutes, 30 minutes, 60 minutes, and 120 minutes post-injection using Siemens Inveon MicroPET/CT Scanner (see FIGS. 17, 18, and Table 2).

TABLE 2
Uptake (SUV) and biodistribution of [⁶⁸Ga]Ga—H₃P₂O₈ in infected tibia,
normal tibia, contralateral muscle, and normal muscle mouse models

| | [⁶⁸Ga]Ga—H₃P₂O₈ - SUV (Average ± SD) | | | | |
|---|---|---|---|---|---|
| | Osteomyelitis Foreign Body model | | | Normal | |
| | Infected | | | Normal | Muscle in |
| Time | Tibia | Contralateral | Muscle | Tibia | Normal |
| (min) | (n = 5) | Tibia (n = 5) | (n = 5) | (n = 4) | group (n = 4) |
| 15 | 1.82 ± 0.57* | 0.76 ± 0.17 | 0.59 ± 0.14^{#} | 0.76 ± 0.03 | 0.35 ± 0.10 |
| 30 | 2.40 ± 0.49* | 0.59 ± 0.12 | 0.58 ± 0.20 | 0.76 ± 0.09 | 0.50 ± 0.12 |
| 60 | 2.87 ± 0.66* | 0.60 ± 0.15 | 0.67 ± 0.18 | 0.80 ± 0.09 | 0.49 ± 0.10 |
| 120 | 3.48 ± 0.25* | 0.29 ± 0.18 | 0.76 ± 0.30 | 0.89 ± 0.16 | 0.48 ± 0.09 |

*Infected Tibia vs Normal Tibia,
^{#}Contralateral Muscle vs Normal Muscle

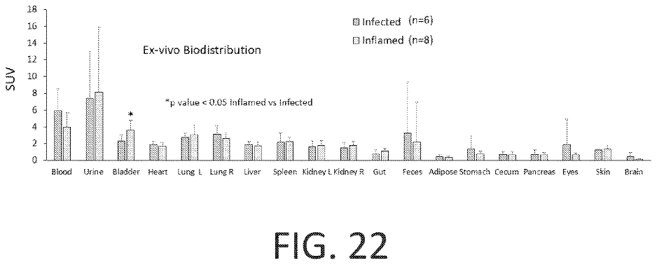

The acquired PET images were visualized and analyzed for quantifying standardized uptake value (SUV) using image analysis software—MIM 7 software and PMOD. Following final imaging at 120 minutes post-injection, the animal was euthanized, and major organs and infected and inflamed tibia were extracted for gamma counting for ex-vivo biodistribution analysis (see FIG. 22). At the end of last imaging session, rats were euthanized by cardiectomy, and the operated tibia with K-wire or contralateral tibia were aseptically removed. Following removal, tibias were weighed, and Ga-68 radioactivity was counted in 2480 Wizard2 automatic gamma counter for ex-vivo biodistribution assessment. The tibias were then frozen to −80° C. The frozen tibia from each animal was crushed aseptically using a bone pulverizer chilled at −80° C. The crushed tibia without K wire was weighed and vortexed in 1 mL sterile saline. The K wire was also vortexed in 1.0 mL sterile saline separately. The vortexed tibia and K-wire saline suspension were sonicated for 5 minutes. After sonication, 1:10 serial dilutions were prepared in saline and 0.1 mL of each dilution was plated in Trypticase Soy Agar plates with 5% Sheep Blood (TSA II). After 24 hours of incubation at 37° C., the bacterial cultures in tibia suspension and K-wire suspension were quantified. In the 2-week-old osteomyelitis model used in the study, the infected tibia contained 7.00±0.19 log₁₀ cfu/g tibia (n=10) and 5.41±0.40 log₁₀ cfu/K-wire (n=10). No bacteria were observed in contralateral bone.

### Conclusions

To assess the preferential uptake of novel [⁶⁸Ga]Ga-H₃P₂O₈ PET probe in infection as compared to inflammation, the same surgery and imaging was performed but omitted the MRSA. The results demonstrated for the first time that PET probe [⁶⁸Ga]Ga-H₃P₂O₈ can be successfully used as a non-invasive imaging probe for in vivo quantification of Staphylococcal aureus infection in an orthopedic surgical implant model even in presence of the inflammation. The probe [⁶⁸Ga]Ga-H₃P₂O₈ was also successfully used to detect thigh infection caused by E coli in a mouse model.

Example 1 illustrates [⁶⁸Ga]Ga-H₃P₂O₈ as a PET probe that enables medical imaging sites of bacterial infection and is easy to produce. The probe uses Ga-68 as a surrogate marker for iron, which bacteria need to thrive. The probe [⁶⁸Ga]Ga-H₃P₂O₈ enables detecting infections including but not limited to infections of post hip or knee replacements, infection of implanted cardiac devices, spine infusions. Additionally or alternatively, the probe [⁶⁸Ga]Ga-H₃P₂O₈ can be used to determine origin of fever of unknown cause.

### Example 2

The inventors have designed and developed a small molecule radiolabeled with positron emitting isotope Ga-68 as [⁶⁸Ga]Ga-H₃P₂O₈, a PET probe to noninvasively image bacterial infection [see FIG. 2]. In this innovative work, the inventors have creatively designed, synthesized and evaluated noninvasive micro PET/CT imaging using a novel PET probe [⁶⁸Ga]Ga-H₃P₂O₈ in osteomyelitis foreign body rat model to locate bacterial infection caused by the Staphylococcus aureus along with infection caused by E coli. The results are outstanding and showed >10-fold higher uptake of [⁶⁸Ga]Ga-H₃P₂O₈ in infected tibia in comparison to the contralateral tibia at 120 minutes post injection of the PET probe. In fact, significant differences were observed in the uptake of [⁶⁸Ga]Ga-H₃P₂O₈ in infected tibia in comparison to the contralateral tibia as early as 15 minutes post injection of the PET probe, and this difference in uptake grew over time. The observed site of bacterial infection with noninvasive PET/CT imaging using [⁶⁸Ga]Ga-H₃P₂O₈ was also corroborated by performing the bone biopsy of the site of infection and culturing the bacteria for their presence. Given the clinical and commercial potentials of the developed PET probe [⁶⁸Ga]Ga-H₃P₂O₈, we envisioned to develop an automated current good manufacturing practice (cGMP) based synthesis of [⁶⁸Ga]Ga-H₃P₂O₈, quality control method validation and additional evaluation of [⁶⁸Ga]Ga-H₃P₂O₈ in other bacterial infections (caused by other gram-negative and gram positive bacteria) for a wider application of the developed technology. The automated cGMP synthesis entails, assurance of the identity, strength, quality, and purity of [⁶⁸Ga]Ga-H₃P₂O₈. This includes establishing a strong quality control method, obtaining appropriate quality of the raw materials, establishing robust operating procedures, detecting, and investigating product quality deviations, and maintaining traceable documentation. In addition, successful imaging of [⁶⁸Ga]Ga-H₃P₂O₈ in additional bacterial strains, such as Pseudomonas aeruginosa and Klebsiella pneumoniae for other applications of the developed [⁶⁸Ga]Ga-H₃P₂O₈ PET probe is envisioned. To the best of our knowledge this is highly novel invention: there is currently no PET probe to noninvasively detect live bacteria and to differentiate between inflammation and infection the way this tracer does.

### Example 3

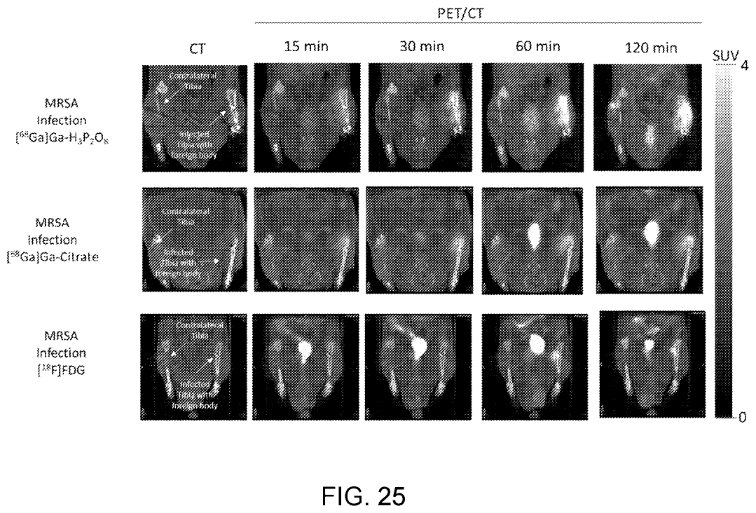

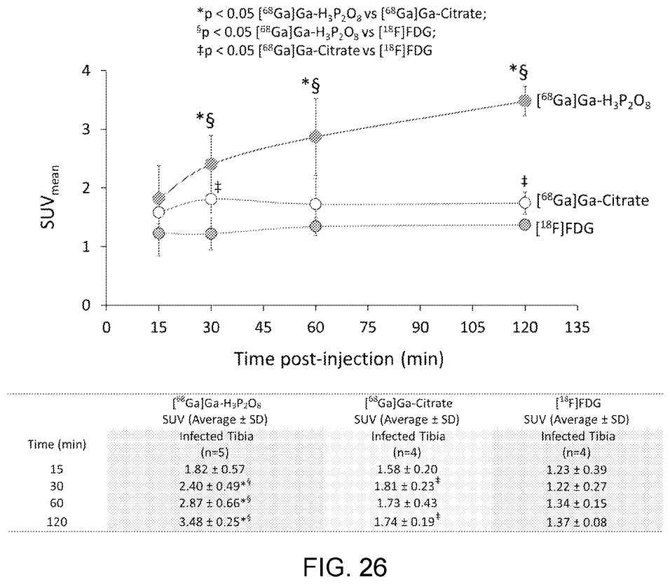

The efficacy of [⁶⁸Ga]Ga-H₃P₂O₈ for imaging infection was compared to other PET imaging probes, [⁶⁸Ga]Ga-Citrate and [¹⁸F]Fluorodeoxyglucose ([¹⁸F]FDG). In a side-by-side study, all three probes were used to image a MRSA infection in an osteomyelitis foreign body rat model (i.e., an infected tibia). Representative PET/CT images are shown in FIG. 25. The bright portions of these images at the infected tibias demonstrate that the [⁶⁸Ga]Ga-H₃P₂O₈ probe localizes at the infected tibia to a greater extent than [⁶⁸Ga]Ga-Citrate or [¹⁸F]FDG. As can be seen in FIG. 26, the change in mean SUV for each of the time points and demonstrates [⁶⁸Ga]Ga-H₃P₂O₈ has a significantly higher mean SUV after 60 minutes post injection than [⁶⁸Ga]Ga-Citrate or [¹⁸F]FDG.

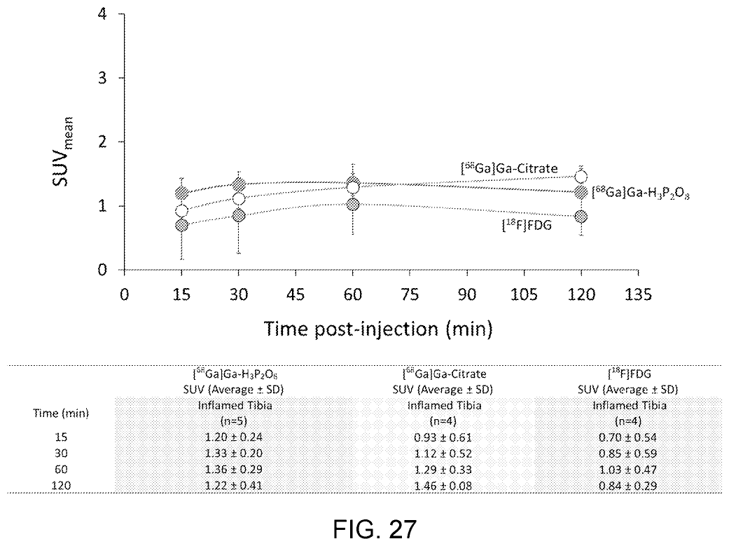

To further investigate the selectivity of [⁶⁸Ga]Ga-H₃P₂O₈ for imaging infection over inflammation, this novel probe was compared to other PET imaging probes, [⁶⁸Ga]Ga-Citrate and [¹⁸F]FDG in an inflammation model. In a side-by-side study, all three probes were used to image inflammation (i.e., an inflamed tibia) in an osteomyelitis foreign body rat model. There is no significant difference in the mean SUV for each probe at each time point up to 120 minutes, as shown in FIG. 27.

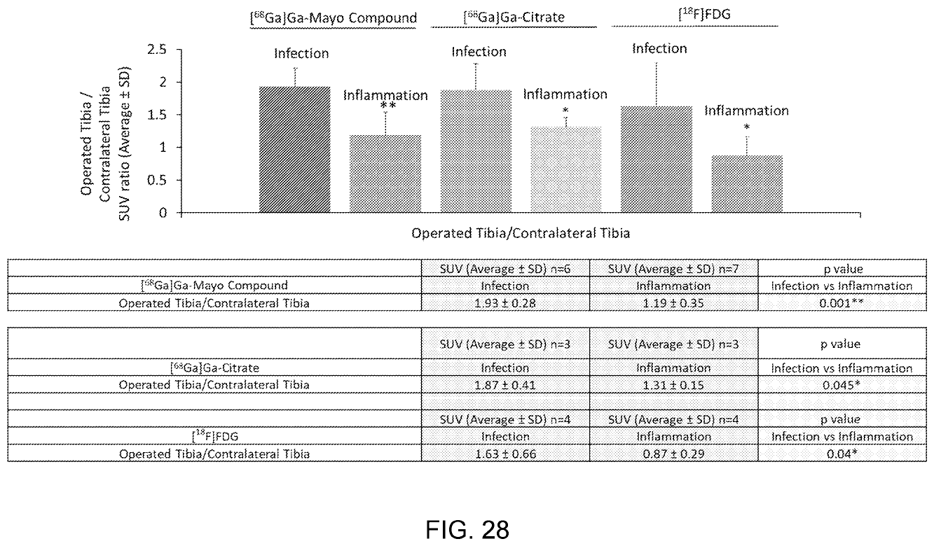

Turning now to FIG. 28, the localization of [⁶⁸Ga]Ga-H₃P₂O₈ at an infection was investigated in comparison to [⁶⁸Ga]Ga-Citrate and [¹⁸F]FDG. The uptake of [⁶⁸Ga]Ga-H₃P₂O₈, [⁶⁸Ga]Ga-citrate and [¹⁸F]FDG at 120 minutes post-injection in areas of Methicillin-resistant Staphylococcus aureus (MRSA) infection was determined by ex vivo biodistribution. Additionally, the uptake of [⁶⁸Ga]Ga-H₃P₂O₈, [⁶⁸Ga]Ga-citrate and [¹⁸F]FDG at 120 minutes post-injection in an inflammation was determined similarly. The uptake at sites of infection and inflammation are expressed as SUV ratios, or the ratio of the SUV of the operated (infected) tibia to the SUV of the contralateral (uninfected) tibia. The SUV ratio data show a significant difference between infection and inflammation for [⁶⁸Ga]Ga-H₃P₂O₈ (p=0.001). In comparison, the SUV ratio data show a less statistically significant difference between infection and inflammation for [⁶⁸Ga]Ga-citrate (p=0.045) and [¹⁸F]FDG (p=0.04), further demonstrating the utility of [⁶⁸Ga]Ga-H₃P₂O₈ to selectively identify infection over inflammation.

### Example 4

Not only can [⁶⁸Ga]Ga-H₃P₂O₈ be used to differentiate infection and inflammation, [⁶⁸Ga]Ga-H₃P₂O₈ may be useful in distinguishing infection by different microbes using PET image analysis.

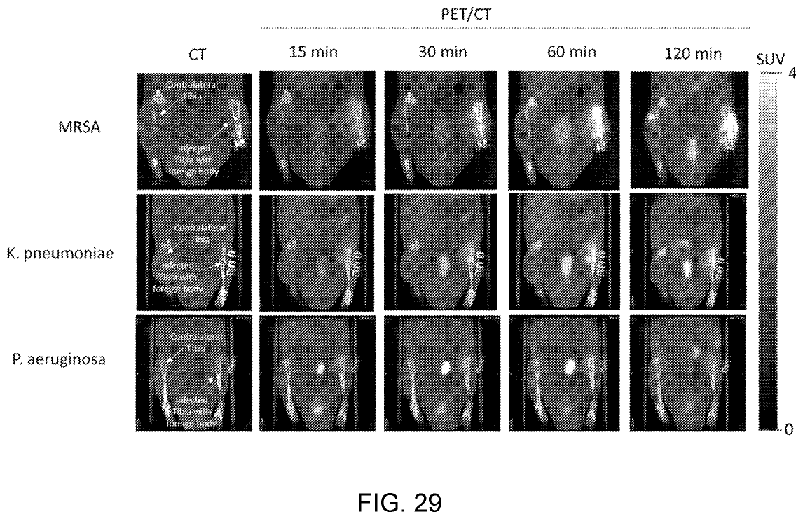

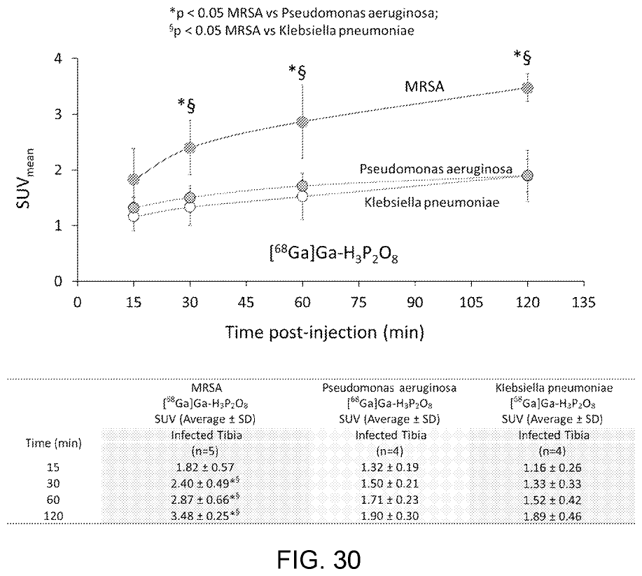

Turning now to FIG. 29, [⁶⁸Ga]Ga-H₃P₂O₈ was used in a study to demonstrate imaging of infection by a variety of microbes. Osteomyelitis foreign body rat models with Methicillin-resistant Staphylococcus aureus (MRSA), Klebsiella pneumoniae (K. pneumoniae), or Pseudomonas aeruginosa (P. aeruginosa) infection were imaged at different time points post-injection (FIG. 29). As can be seen in FIG. 30, the mean SUV for the MRSA-infected animal is significantly higher (p<0.05) than the mean SUV for the animals infected with K. pneumoniae or P. aeruginosa.

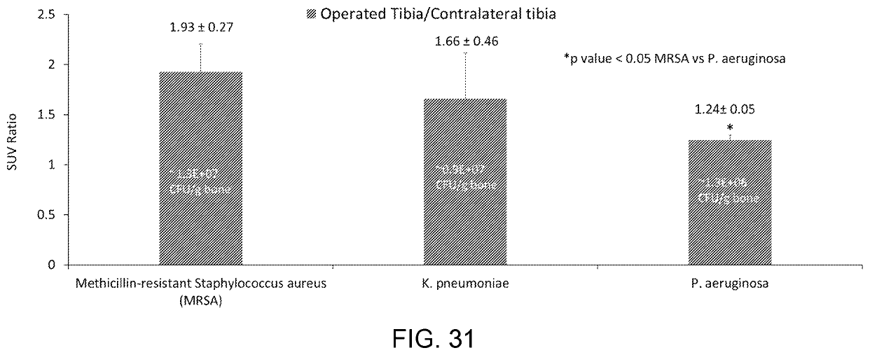

On further investigation, the uptake of [⁶⁸Ga]Ga-H₃P₂O₈ in rat models infected by different microbes was determined. Osteomyelitis foreign body rat models were infected with Methicillin-resistant Staphylococcus aureus (MRSA), Klebsiella pneumoniae (K. pneumoniae) or Pseudomonas aeruginosa (P. aeruginosa). The infected, operated tibias were compared to uninfected, contralateral tibias by ex vivo biodistribution. The uptake shown in FIG. 31 is expressed as SUV ratios (Operated tibia/contralateral tibia) and show a significant difference in uptake between MRSA and P. aeruginosa (p<0.05).

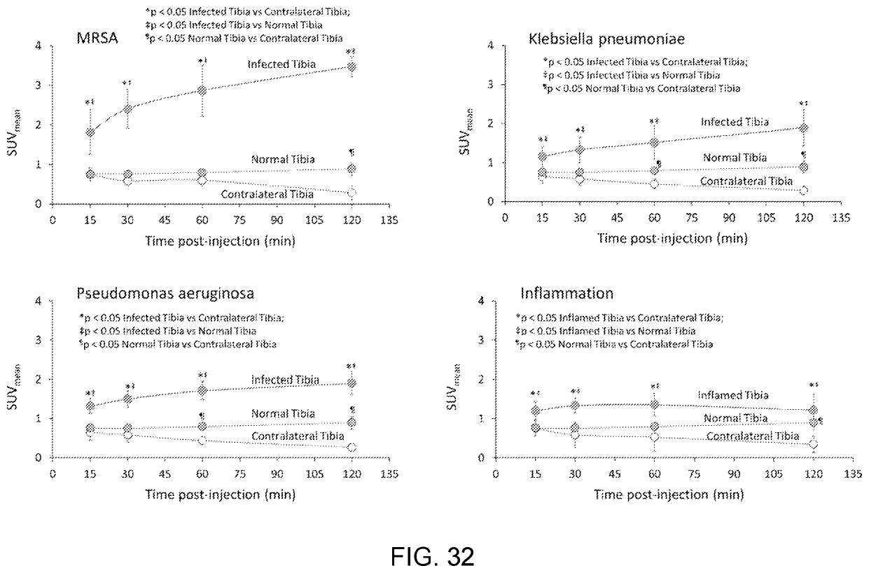

A comparison of rat models infected with MRSA, K. pneumoniae, or P. aeruginosa was undertaken and the results shown in FIG. 32 using [⁶⁸Ga]Ga-H₃P₂O₈ as the PET probe. The mean SUV of infected tibia vs contralateral tibia, inflamed tibia and normal tibia in uninfected rat at different time points post-injection as determined by PET image analysis.

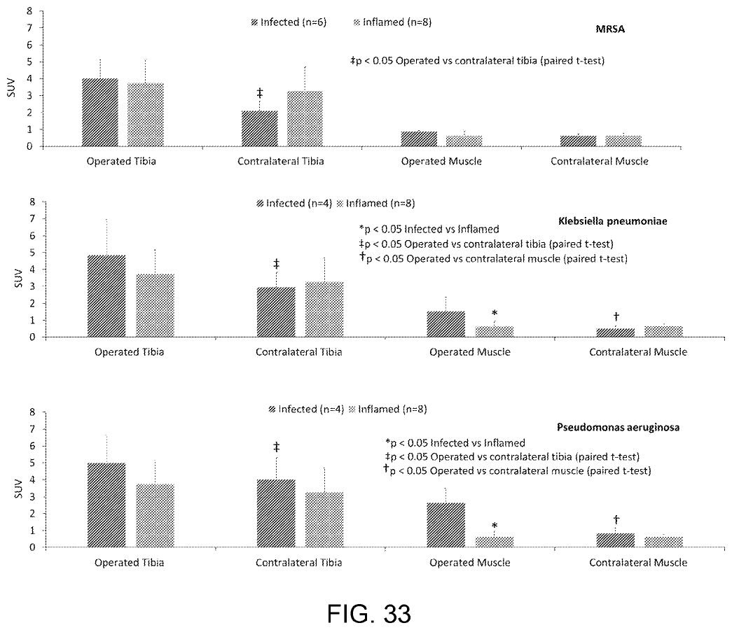

FIG. 33 shows a comparison of the SUV of [⁶⁸Ga]Ga-H₃P₂O₈ in infected tibia, contralateral tibia, infected muscle, contralateral muscle in an osteomyelitis foreign body rat model with Methicillin-resistant Staphylococcus aureus (MRSA) or Klebsiella pneumoniae (K. pneumoniae) or Pseudomonas aeruginosa (P. aeruginosa) infection at different time points post-injection as determined by the ex vivo biodistribution at 120 minutes post-injection.

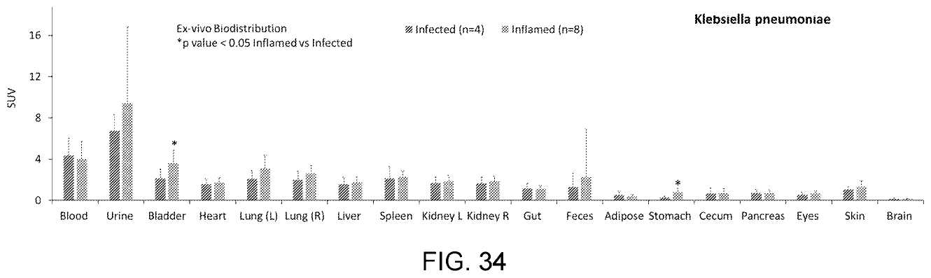

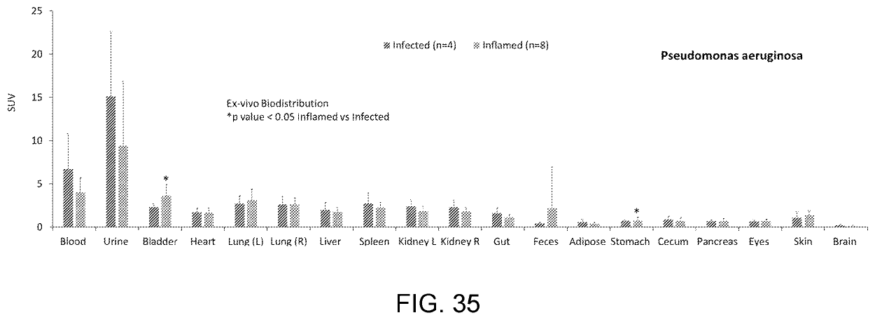

Ex vivo biodistribution was undertaken to determine if the [⁶⁸Ga]Ga-H₃P₂O₈ probe localized in any organs or excrement of the animal (i.e., blood, urine, bladder, heart, lungs, liver, spleen, kidneys, gut, feces, adipose, stomach, cecum, pancreas, eyes, skin, and brain). FIG. 34 shows a comparison of the SUV of [⁶⁸Ga]Ga-H₃P₂O₈ in an osteomyelitis foreign body rat model with either K. pneumoniae infection (FIG. 34) or P. aeruginosa (FIG. 35) and in the inflammation rat models at 120 minutes post-injection. In general, the distribution of [⁶⁸Ga]Ga-H₃P₂O₈ in the infected models was similar to the distribution in the inflammation model, with comparable SUVs for each organ/excrement except for the bladder and feces in both K. pneumoniae and P. aeruginosa.

FIG. 35 shows a comparison of the SUV of [⁶⁸Ga]Ga-H₃P₂O₈ in an osteomyelitis foreign body rat model with Pseudomonas aeruginosa (P. aeruginosa) infection and in the inflammation rat models as determined by the ex vivo biodistribution at 120 minutes post-injection.

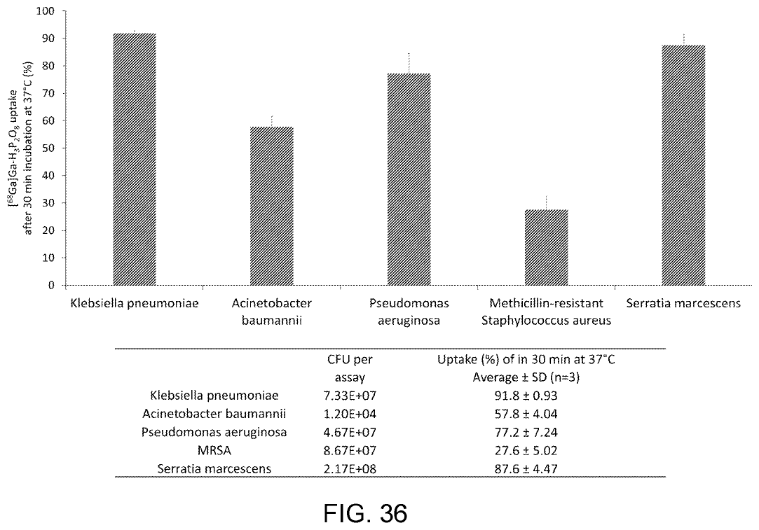

The in vitro uptake of [⁶⁸Ga]Ga-H₃P₂O₈ in different bacterial strains cultured in iron deficient T-medium is shown in FIG. 36. The uptake of [⁶⁸Ga]Ga-H₃P₂O₈ in bacteria was measured after 30 minutes incubation with [⁶⁸Ga]Ga-H₃P₂O₈ in an iron deficient T-medium at 37° C. using standard uptake calculation using following formula: Uptake in bacterial pellet=(Decay corrected ⁶⁸Ga radioactivity in bacterial pellet fraction (after incubation and washes)÷Decay corrected total ⁶⁸Ga radioactivity in initial bacterial suspension (at the start of incubation)×100. The radioactivity in bacterial suspension or bacterial pellet for calculation of uptake was decay corrected to time when incubation was started.

### REFERENCES

1. Cook G E et al., Infection in Orthopaedics. J Orthop Trauma. 2015 December; 29 Suppl 12:S19-23.
2. Wolford M L et al., Hospitalization for total hip replacement among inpatients aged 45 and over: United States, 2000-2010. NCHS Data Brief. 2015 February; (186):1-8.
3. Sethi I et al., Current Status of Molecular Imaging of Infection: A Primer. AJR Am J Roentgenol. 2019 August; 213(2):300-308.
4. Rabin N et al., Biofilm formation mechanisms and targets for developing antibiofilm agents. Future Med Chem. 2015; 7(4):493-512.
5. Vestby L K et al., Bacterial Biofilm and its Role in the Pathogenesis of Disease. Antibiotics (Basel). 2020 Feb. 3; 9(2):59.
6. Signore A, et al., Imaging Bacteria with Radiolabelled Probes: Is It Feasible? J Clin Med. 2020 Jul. 25; 9(8):2372
7. Palestro C J., Radionuclide Imaging of Musculoskeletal Infection: A Review. J Nucl Med. 2016 September; 57(9):1406-12.
8. Gowrishankar G et a., Investigation of 6-[¹⁸F]-fluoromaltose as a novel PET tracer for imaging bacterial infection. PLoS One. 2014 Sep. 22; 9(9):e107951.
9. Weinstein E A et al., Imaging Enterobacteriaceae infection in vivo with 18F-fluorodeoxysorbitol positron emission tomography. Sci Transl Med. 2014 Oct. 22; 6(259):259ra146.
10. Pieracci F M et al., Iron and the risk of infection. Surg Infect (Larchmt). 2005; 6 Suppl 1:S41-6.
11. Williams P et al., Bacterial transferrin receptors: Structure, function and contribution to virulence. Med Microbiol Immunol 1992; 181:301-322.
12. Ioppolo J A et al., 67Ga-labeled deferoxamine derivatives for imaging bacterial infection: Preparation and screening of functionalized siderophore complexes. Nucl Med Biol. 2017 September; 52:32-41.
13. Tseng J R et al., Potential usefulness of 68Ga-citrate PET/CT in detecting infected lower limb prostheses. EJNMMI Res. 2019 Jan. 3; 9(1):2.
14. Petrik M et al., Imaging of Pseudomonas aeruginosa infection with Ga-68 labelled pyoverdine for positron emission tomography. Sci Rep. 2018 Oct. 24; 8(1):15698.
15. Joaqui-Joaqui M A et al., Catechol-Based Functionalizable Ligands for Gallium-68 Positron Emission Tomography Imaging. Inorg Chem. 2020 Sep. 8; 59(17):12025-12038.
16. Lankinen P et al., A Comparative ⁶⁸Ga-Citrate and ⁶⁸Ga-Chloride PET/CT Imaging of Staphylococcus aureus Osteomyelitis in the Rat Tibia. Contrast Media & Molecular Imaging. 2018 Jan. 18; 2019:9892604.
17. Brinkman C L et al., A novel rat model of foreign body osteomyelitis for evaluation of antimicrobial efficacy. J Exp Appl Anim Sci. 2019; 3(1):7-14.

The citation of any document or reference is not to be construed as an admission that it is prior art with respect to the present invention.

Thus, the invention provides positron emission tomography imaging probes and their use in medical imaging of bacterial infection and inflammation. In particular, the present invention describes the PET imaging probe, [⁶⁸Ga]Ga-H₃P₂O₈, for the noninvasive imaging of bacterial infection. The PET probe has shown significantly higher uptake (SUV) in mouse model of thigh infection as well as in in-vitro studies and also in osteomyelitis foreign body rat model to locate bacterial infection caused by the Staphylococcus aureus along with infection caused by E coli. Our results are outstanding and showed >10-fold higher uptake of [⁶⁸Ga]Ga-H₃P₂O₈ in infected tibia in comparison to the contralateral tibia at 120 minutes post injection of the PET probe. In fact, our developed probe preferentially distinguishes between bacterial infection over inflammation which is an inherent challenge in surgical patients with implants including orthopedic implants, spine infusion, cardiac implants, and fevers of unknown origin.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment,” or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another, unless indicated otherwise.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A composition comprising:
a radiopharmaceutical including:
(i) cations comprising a positron emitter, and
(ii) anions selected from the group consisting of [PO₄]³⁻, [HPO₄]²⁻, [H₂PO₄]⁻, and mixtures thereof,
wherein the cations are ionically bonded to the anions.

2. The composition of claim 1 wherein:
the positron emitter is selected from the group consisting of ¹¹C, ¹³N, ¹⁵O, ¹⁸F, ^{34m}Cl, ³⁸K, ⁴³Sc, ⁴⁴Sc, ⁴⁵Ti, ⁵¹Mn, ⁵²Mn, ^{52m}Mn, ⁵²Fe, ⁵³Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ^{189/189m}Hg, ¹⁹¹Hg, ^{191m}Hg, ¹⁹³Hg, ^{193m}Hg, ¹⁹⁵Hg, ^{195m}Hg, ⁷⁵Br, ⁷⁶Br, ⁸²Rb, ⁸⁶Y ⁸⁹Zr, ⁹⁰Nb, ^{94m}Tc, ^{110m}In, ¹¹⁵Sb, ^{116m2}Sb, ^{118m2}Sb, ¹¹⁸Sb, ¹²⁰Sb, and ¹²⁴I.

3. The composition of claim 1 wherein:
the positron emitter is ⁶⁸Ga.

4. The composition of claim 3 wherein:
the radiopharmaceutical has the formula: [⁶⁸Ga]Ga-H₃P₂O₈.

5. The composition of claim 3 wherein:
the radiopharmaceutical has the following structure:

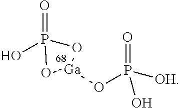

6. The composition of claim 1 further comprising:
at least one of phosphate buffer, potassium chloride, sodium chloride, and mixtures thereof.

7. The composition of claim 1 wherein:
a pH of the composition is in a range of 5 to 7.

8. The composition of claim 1 wherein:
the radiopharmaceutical is adapted for targeting a site of bacterial infection.

9. The composition of claim 1 wherein:
the radiopharmaceutical is adapted for targeting a site of one of both of gram-negative and gram-positive bacterial infection of any organ or tissue.

10. The composition of claim 1 wherein:
the radiopharmaceutical is adapted for targeting a site of one or both of gram-negative and gram-positive bacterial infection of bone, muscle, heart, liver, lung, vascular grafts, interventional grafts, vascular graft infections or foreign body surgical implants.

11. The composition of claim 1 wherein:
a detectable amount of the radiopharmaceutical is present in the composition, and
the detectable amount of the radiopharmaceutical is an amount of the radiopharmaceutical that is sufficient to enable detection of accumulation of the radiopharmaceutical in cells or tissue or an organ of interest at a site of bacterial infection by a medical imaging technique.

12. The composition of claim 11 wherein:
the medical imaging technique is selected from positron emission tomography imaging, positron emission tomography with computed tomography imaging, positron emission tomography with magnetic resonance imaging, or positron emission tomography with single-photon emission computed tomography.

13. The composition of claim 11 wherein:
the medical imaging technique is positron emission tomography imaging.

14. The composition of claim 11 wherein:
the site of bacterial infection is a bone or adjacent to a bone or graft infection.

15. The composition of claim 11 wherein:
the site of bacterial infection is a femur or a thigh bone or a bacterial infection related to orthopedic surgical implants.

16. The composition of claim 11 wherein:
the site of bacterial infection is a surgical implant selected from spine infusion, a hip replacement, and a knee replacement.

17. The composition of claim 11 wherein:
the bacterial infection is a result of a gram-negative bacteria.

18. The composition of claim 11 wherein:
the bacterial infection is a result of Escherichia coli.

19. The composition of claim 11 wherein:
the bacterial infection is a result of a gram-positive bacteria.

20. The composition of claim 11 wherein:
the bacterial infection is a result of Staphylococcus aureus.

21. A method for in vivo imaging of a subject, the method comprising:
(a) administering to the subject the composition of any of claims 1-20;
(b) waiting a time sufficient to allow the radiopharmaceutical to accumulate at a tissue or cell site or an organ of interest to be imaged; and
(c) imaging the cells or tissues or the organ of interest with a medical imaging technique.

22. The method of claim 21 wherein:
the medical imaging technique is selected from positron emission tomography imaging, positron emission tomography with computed tomography imaging, positron emission tomography with magnetic resonance imaging, or positron emission tomography with single-photon emission computed tomography.

23. A method of imaging a subject by positron emission tomography, the method comprising:
(a) administering the composition of any of claims 1-20 to the subject;
(b) using a plurality of detectors to detect gamma rays emitted from the subject and to communicate signals corresponding to the detected gamma rays; and
(c) reconstructing from the signals a series of medical images of a region of interest of the subject.

24. An imaging method comprising acquiring an image of a subject to whom a detectable amount of the radiopharmaceutical of the composition of any of claims 1-20 has been administered.

25. The method of claim 24, which comprises acquiring an image of a region of bacterial infection in a bone or adjacent to a bone in a muscle of the subject.

26. The method of claim 25, wherein the region of bacterial infection is in or adjacent to a femur, a thigh bone, or a surgical implant of the subject.

27. The method of claim 24, which comprises acquiring an image of a region of a heart or adjacent to a heart of the subject.

28. The method of claim 24, which comprises acquiring an image of a region of bacterial infection related to myocardial infection or a cardiac device.

29. The method of claim 24, which comprises acquiring an image of a region of bacterial infection related to a pacemaker.

30. The method of claim 24, which comprises acquiring an image of a region of a joint or adjacent to a joint of the subject.

31. The method of claim 30, wherein the joint is arthritic or includes an orthopedic surgical implant.

32. The method of claim 24, which comprises acquiring the image using positron emission tomography imaging, positron emission tomography with computed tomography imaging, positron emission tomography with magnetic resonance imaging, or positron emission tomography with single-photon emission computed tomography.

33. The method of claim 24, wherein the detectable amount of the radiopharmaceutical is an amount of the radiopharmaceutical that is sufficient to enable detection of accumulation of the radiopharmaceutical in cells or tissue or an organ of interest by a medical imaging technique.

34. A method for detecting bacterial infection in a subject, the method comprising:
(a) administering to the subject the composition of any of claims 1-20;
(b) waiting a time sufficient to allow the radiopharmaceutical to accumulate at a tissue or cell site or an organ of interest to be imaged; and
(c) imaging the cells or tissues or the organ of interest with a medical imaging technique.

35. The method of claim 34 further comprising (d) comparing a data set from the image of the cells or tissues or the organ of interest to a previously acquired data set from an image of a known infection.

36. The method of claim 34 wherein:
the bacterial infection is a result of a gram-negative bacteria.

37. The method of claim 34 wherein:
the bacterial infection is a result of Escherichia coli.

38. The method of claim 34 wherein:
the bacterial infection is a result of a gram-positive bacteria.

39. The method of claim 34 wherein:
the bacterial infection is a result of Staphylococcus aureus.

40. The method of claim 34 wherein:
the tissue or cell site is a bone or adjacent to a bone or grafts.

41. The method of claim 40 wherein:
the bone is a femur or a thigh of the subject.

42. The method of claim 34 wherein:
the medical imaging technique is selected from positron emission tomography imaging, positron emission tomography with computed tomography imaging, positron emission tomography with magnetic resonance imaging, or positron emission tomography with single-photon emission computed tomography.

43. The method of claim 34 wherein:
step (b) comprises allowing the radiopharmaceutical to accumulate at a site of bacterial infection.

44. The method of claim 34 wherein:
step (b) comprises allowing the radiopharmaceutical to accumulate at a site of bacterial infection including gram-positive bacteria.

45. The method of claim 34 wherein:
step (b) comprises allowing the radiopharmaceutical to accumulate at a site of bacterial infection including gram-negative bacteria.

46. A method for detecting or ruling out a condition involving a bacterial infection in a subject, the method comprising:
(a) administering to a subject the composition of any of claims 1-20 wherein the radiopharmaceutical is targeted to a bacterial infection at a tissue or cell site or an organ of interest in the subject; and
(b) acquiring an image of the cells or tissues or the organ of interest to detect the presence or absence of a bacterial infection in the subject.

47. The method of claim 46 further comprising (c) comparing a data set from the image of the cells or tissues or the organ of interest to a previously acquired data set from an image of a known infection.

48. The method of claim 46 further comprising (c) determining an SUV from the image of the cells or tissues or the organ of interest and comparing the SUV to a previously acquired SUV from an image of a known infection.

49. The method of claim 46 wherein:
step (b) comprises acquiring the image using a medical imaging technique selected from positron emission tomography imaging, positron emission tomography with computed tomography imaging, positron emission tomography with magnetic resonance imaging, and positron emission tomography with single-photon emission computed tomography.

50. The method of claim 46 wherein:
step (b) comprises acquiring an image of a region of bacterial infection in a bone or adjacent to a bone in a muscle of the subject.

51. The method of claim 50, wherein the region of bacterial infection is in or adjacent to a femur, a thigh bone, or a surgical implant of the subject.

52. The method of claim 46 wherein:
step (b) comprises acquiring an image of a region of a heart or adjacent to a heart of the subject.

53. The method of claim 46 wherein:
step (b) comprises acquiring an image of a region of bacterial infection related to myocardial infection or a cardiac device.

54. The method of claim 46 wherein:
step (b) comprises acquiring an image of a region of bacterial infection related to a pacemaker.

55. The method of claim 46 wherein:
step (b) comprises acquiring an image of a region of a joint or adjacent to a joint of the subject.

56. The method of claim 55, wherein the joint is arthritic or includes an orthopedic surgical implant.

57. The method of claim 46 wherein:
the bacterial infection is a result of a gram-negative bacteria.

58. The method of claim 46 wherein:
the bacterial infection is a result of Escherichia coli.

59. The method of claim 46 wherein:
the bacterial infection is a result of a gram-positive bacteria.

60. The method of claim 46 wherein:
the bacterial infection is a result of Staphylococcus aureus.

61. The method of claim 46 wherein:
the method can differentiate between inflammation and the bacterial infection.

62. The method of claim 46 wherein:
the cation conjugates with ferritin and/or transferrin and/or lactoferrin at the tissue or cell site.

63. A method for detecting or ruling out a condition involving inflammation in a subject, the method comprising:
(a) administering to a subject the composition of any of claims 1-20 wherein the radiopharmaceutical is targeted to a site of inflammation in a tissue or cell site or an organ of interest in the subject; and
(b) acquiring an image of the cells or tissues or the organ of interest to detect the presence or absence of an inflammation.

64. The method of claim 63 further comprising (c) comparing a data set from the image of the cells or tissues or the organ of interest to a previously acquired data set from an image of a known inflammation.

65. The method of claim 63 further comprising (c) determining an SUV from the image of the cells or tissues or the organ of interest and comparing the SUV to a previously acquired SUV from an image of a known inflammation.

66. The method of claim 63 wherein:
the inflammation is chronic inflammation, neurological inflammation, or inflammation in extremities of the subject.

67. The method of claim 63 wherein:
step (b) comprises acquiring the image using a medical imaging technique selected from positron emission tomography imaging, positron emission tomography with computed tomography imaging, positron emission tomography with magnetic resonance imaging, and positron emission tomography with single-photon emission computed tomography.

68. The method of claim 63 wherein:
step (b) comprises acquiring an image of a region of inflammation in a bone or adjacent to a bone in a muscle of the subject or in a graft or adjacent to the graft.

69. The method of claim 68, wherein the region of inflammation is in or adjacent to a femur, a thigh bone, or a surgical implant of the subject.

70. The method of claim 63 wherein:
step (b) comprises acquiring an image of a region of a heart or adjacent to a heart of the subject.

71. The method of claim 63 wherein:
step (b) comprises acquiring an image of a region of inflammation related to myocardial infection or a cardiac device.

72. The method of claim 71 wherein:
step (b) comprises acquiring an image of a region of inflammation related to a pacemaker.

73. The method of claim 63 wherein:
step (b) comprises acquiring an image of a region of a joint or adjacent to a joint of the subject.

74. The method of claim 73, wherein the joint is arthritic or includes an orthopedic surgical implant.
